SEQUENCE LISTING (43 sequences)

SEQ ID NO: 1 - DNA, 211 bp, Sus scrofa
tgaagaaactttagatgtttcaacaactccatgagcaatttacttaaatcaagatgttgctggtcttggtttaagtgataaacaattagaaagagaaggagttgattttgtttcagttgaaattcctgcttcttcacttccaagagctcatgcagataacttagaaaaacaattttcatttttaaaaatgaatgtagaaaaatcaacagga

SEQ ID NO: 2 - DNA, 223 bp, Mus musculus
gaattcgaaaatgaagaaactttagatgtttcaacaactccatgagcaatttacttaaatcaagatgttgctggtcttggtttaagtgataaacaattagaaagagaaggagttgattttgtttcagttgaaattcctgcttcttcacttccaagagctcatgcagataacttagaaaaacaattttcatttttaaaaatgaatgtagaaaaatcaacaggaa

SEQ ID NO: 3 - DNA, 211 bp, Mus musculus; identical to SEQ ID NO: 1

SEQ ID NO: 4 - DNA, 130 bp, Sus scrofa
ctgtagctgcaaaaaatccttcatatgtgaagaaaattgctgctaatgataaaacaaaaccaaagcctggaattgtatattttaaactttctttgttttctacgatttctttaatatctacaaaaactgg

SEQ ID NO: 5 - DNA, 140 bp, Mus musculus
ctgtagctgcaaaaaatccttcatatgtgaagaaaattgctgctaatgataaaacaaaaccaaagcctggaattgtatattttaaactttctttgttttctacgatttctttaatatctacaaaaactggttttgaattc

SEQ ID NO: 6 - DNA, 130 bp, Mus musculus; identical to SEQ ID NO: 4

SEQ ID NO: 7 - DNA, 184 bp, Sus scrofa
gataatacaagaagtggtttattgcttcaagtaaaaaggattttaagcaaaatcaagacagattacccaaaactttattgatggaaaatgttaaatcattaacaaataaaaaatttatgtctcaatttgaagattgaattaaatttttagaaactttaggatataactcgagttggaaagtttt

SEQ ID NO: 8 - DNA, 190 bp, Mus musculus
gataatacaagaagtggtttattgcttcaagtaaaaaggattttaagcaaaatcaagacagattacccaaaactttattgatggaaaatgttaaatcattaacaaataaaaaatttatgtctcaatttgaagattgaattaaatttttagaaactttaggatataactcgagttggaaagttttgaattc

SEQ ID NO: 9 - DNA, 184 bp, Mus musculus; identical to SEQ ID NO: 7

SEQ ID NO: 10 - DNA, 510 bp, Sus scrofa
atttggattttgcaattgcacttcctgcaataaatgtagctgcatttagtaaaaatacaacaaaattagaattagcagttcaaaatatgtcacaatttcaaaaaggagcttatactggcgaaatttctgcgcaaatgttattggatttaaatgtcaaatatgctattattggtcattcagaaagaaggcaatattttaaagaaactgatttagatgtaaatgcaaaaacacaacaagcaattaaaataatattattccagttgtttgtgtgggtgaaacattagaagaatatgaagcacaaaaaacaagtcaagtaattgaatatcaactaaaaaattcattaaaagatgtagatttaagcaaagttatagtggcttacgaaccaatttgagcaattggtacaggaaaaactgctacaccagagcaagcacaacaagtttgcaaatttattagaaaacaaacaaataaaaatttaactatcttatatggtggctcagtttcgcaagaaaa

SEQ ID NO: 11 - DNA, 510 bp, Mus musculus; identical to SEQ ID NO: 10

SEQ ID NO: 12 - DNA, 141 bp, Homo sapiens
ttccagttgtttgtgtgggtgaaacattagaagaatatgaagcacaaaaaacaagtcaagtaattgaatatcaactaaaaaattcattaaaagatgtagatttaagcaaagttatagtggcttacgaaccaatttgagcaa

SEQ ID NO: 13 - DNA, 506 bp, Mus musculus
attaagctatgcagtcggaacgtgggtcataacgtccacgctcgcgagggaaacaacccagatcgtcagctaaggtcccaaaattgtgttaagtgagaaaggttgtgagatttcataaacaactaggaagttggcttagaagcagccaccttttaaagagtgcgtaattgctcactagtcaagagatcttgcgccaataatgtaacgggactcaaacacaataccgaagctacgggcacattatgtgcgttaggagagcgttttaatttcgttgaagtcagaccgtgaggactggtggagagattaaaagtgagaatgccggcatgagtaacgattcgaagtcagaatcttcgacgcctattgggaaaggtttcctgggcaaggttcgtccacccagggttagtcagggcctaagatgaggcagaaatgcatagtcgatggacaacaggttaatattccttgcatgcctgcaggtccactctagaggatccccgggtaccgagctc

SEQ ID NO: 14 - DNA, 195 bp, Mus musculus
agaaaaaactgaaaacacacaacaaagtgaagctccaggaacaaaaactgaaaacacacaacaaagtgaagctccaggaacaaacactggaaacaaaacaactagtgaatcaaattctggatcatctacaggtagtcaagcaggaacaactacaaatacaggtagtggatcaaattctgaatcaggaatgaattc

SEQ ID NO: 15 - DNA, 216 bp, Mus musculus
atcaggatcaacaaatggaactgctggaacaaattcatcaggttcaactaatggttcaggacaaagtgaagctccaggaacaaacactggaaacaaaacaactagtgaatcaaattctggatcatctacaggtagtcaagcaggaacaactacaaatacaggtagtggatcaaattctgaatcaggaatgaattccaaatacttaataatgttgct

SEQ ID NO: 16 - DNA, 244 bp, Homo sapiens
acgacgtcaaatcatcatgcctcttacgagtggggcaacacacgtgctacaatggtcggtacaaagagaagcaatatggtgacatggagcaaatctcaaaaaaccgatctcagttcggattgaagtctgcaactcgacttcatgaagtcggaatcgctagtaatcgtagatcagctacgctacggtgaatacgttctcgggttttgtacacaccgcccgtcacaccatgggagttggtaatgcc

SEQ ID NO: 17 - DNA, 354 bp, Mus musculus
gttaagtcctgcaacgagcgcaacccttttctttagttactaatattaagttaaggactctagagatactgcctgggtaaccaggaggaaggtggggaccgacgtcaaatcatcatgcctcttacgagtggggcaacacacgtgctacaatggtcggtacaaagagaagcaatatggtgacatggagcaaatctcaaaaaaccgatctcagttcggattgaagtctgcaactcgacttcatgaagtcggaatcgctagtaatcgtagatcagctacgctacggtgaatacgttctcgggttttgtacacaccgcccgtcacaccatgggagttggtaatgcccaaagtcggttt

SEQ ID NO: 18 - DNA, 354 bp, Sus scrofa; identical to SEQ ID NO: 17

SEQ ID NO: 19 - DNA, 164 bp, Mus musculus
gaaaaaactgaaaacacacaacaaagtgaagctccaggaacaaacactggaaacaaaacaactagtgaatcaaattctggatcatctacaggtagtcaagcaggaacaactacaaatacaggtagtggatcaaattctgaatcaggaatgaattcgtaatcatg

SEQ ID NO: 20 - DNA, 887 bp, Sus scrofa
ggcttggtgctgctcaaatggatggtgctatcttagttgttgctgcaacagatggaccaatgcctcaaacaagagaacacatactattgtcaaaacaagttggtgttccaaaaatcgttgtattcttaaacaaagtagatatgttacaaggtgaagaagaaatggttgacttagttgaagttgaagttagagaattattaagctcatatgactttgacggagacaatactcctattgttcgtggatctgctaaaggtgctttagaaggaaaaccagaatgagaagcaaaaattcttgaattaatggatgcagttgatacttacattgactcaccagttagagaattagacaaaccattcttaatggcagttgaagacgtttcacaattacaggtagaggaacagttgctacaggtaaagttgaaagaggacaagttaaattaaacgaagaagttgaaattgttggatataaatcagaacctaaaaaaacagttgttacaggtattgaaatgttcaacaaaaacttacaatcagcaatggctggagataatgccggagttcttttaagaggagttaacagagacgaaatcgaaagaggacaagttattgctaaacctaaaactattgttccacatacaacatttaaagcagctatttatgttcttaaaaaagaagaaggtggacgtcacacaccattcttccctaactacaaacctcaattttacttcagaacaactgacgtaacaggtggagttaaatttgaacaaggtgtagaaatggttaaacctggggacaatgtaaatttaactgtagaattaattgctccaattgctgttgaacaaggtactaagttctcaatccgtgaaggtggtagaactgtaggaagcc

SEQ ID NO: 21 - DNA, 882 bp, Mus musculus
ggcttggtgctgctcaaatggatggtgctatcttagttgttgctgcaacagacggaccaatgcctcaaacaagagaacacatactattgtcaaaacaagttggtgttccaaaaatcgttgtattcttaaacaaagtagatatgttacaaggtgaagaagaaatggttgacttagttgaagttgaagttagagaattattaagctcatatgactttgacggagacaatactcctattgttcgtggatctgctaaaggtgctttagaaggaaaaccagaatgagaagcaaaaattcttgaattaatggatgcagttgatacttacattgactcaccagttagagaattagacaaaccattcttaatggcagttgaagacgtttcacaattacaggtagaggaacagttgctacaggtaaagttgaaagaggacaagttaaattaaacgaagaagttgaaattgttggatataaagcagaacctaaaaaaacagttgttacaggtattgaaatgttcaacaaaaacttacaatcagcaatggctggagataatgccggagttcttttaagaggagttaacagagacgaaatcgaaagaggacaagttattgctaaacctaaaactattgttccacatacaacatttaaagcagctatttatgttcttaaaaaagaagaaggtggacgtcacacaccattcttccctaactacaaacctcaattttacttcagaacaactgacgtaacaggtggagttaaatttgaacaaggtgtagaaatggttaaacctggggacaatgtaaatttaactgtagaattaattgctccaattgctgttgaacaaggtactaagttctcaatccgtgaaggtggtagaactgtagg

SEQ ID NO: 22 - DNA, 239 bp, Homo sapiens
ggtgctgctcaaatggatggtgctatcttagttgttgctgcaacagatggaccaatgcctcaaacaagagaacacatactattgtcaaaacaagttggtgttccaaaaatcgttgtattcttaaacaaagtagatatgttacaaggtgaagaagaaatggttgacttagttgaagttgaagttagagaattattaagctcatatgactttgacggagacaatactcctattgttcgtgg

SEQ ID NO: 23 - DNA, 16 bp, Mycoplasma hyorhinis
uutctucuuugttgtt

SEQ ID NO: 24 - DNA, 18 bp, Mycoplasma hyorhinis
ttttttugguugtutucu

SEQ ID NO: 25 - DNA, 19 bp, Mycoplasma hyorhinis
uuutuuguugttcuttttt

SEQ ID NO: 26 - DNA, 19 bp, Mycoplasma hyorhinis
gctttuuugucgcgtttuc

SEQ ID NO: 27 - DNA, 20 bp, Mycoplasma hyorhinis
uuuuttuuugcuucttcugt

SEQ ID NO: 28 - DNA, 20 bp, Mycoplasma hyorhinis
ttugtttuugucctugtugu

SEQ ID NO: 29 - DNA, 19 bp, Mycoplasma hyorhinis
tuucttcugucgttgugct

SEQ ID NO: 30 - DNA, 20 bp, Mycoplasma hyorhinis
uugucctugutgtccutcug

SEQ ID NO: 31 - DNA, 22 bp, Mycoplasma hyorhinis
gtuuutucuuguuttttttctt

SEQ ID NO: 32 - DNA, 20 bp, Artificial Sequence
  Primer
ggttaagtcctgcaacgagc

SEQ ID NO: 33 - DNA, 20 bp, Artificial Sequence
  Primer
gttaactcaccgactttggg

SEQ ID NO: 34 - DNA, 22 bp, Artificial Sequence
  Primer
ggcttggtgctgctcaaatgga

SEQ ID NO: 35 - DNA, 24 bp, Artificial Sequence
  Primer
cctacagttctaccaccttcacgg

SEQ ID NO: 36 - DNA, 25 bp, Artificial Sequence
  Primer
gataatacaagaagtggtttattgc

SEQ ID NO: 37 - DNA, 26 bp, Artificial Sequence
  Primer
aaaactttccaactcgagttatatcc

SEQ ID NO: 38 - DNA, 30 bp, Artificial Sequence
  Primer
tgaagaaactttagatgtttcaacaactcc

SEQ ID NO: 39 - DNA, 22 bp, Artificial Sequence
  Primer
tcctgttgatttttctacattc

SEQ ID NO: 40 - DNA, 28 bp, Artificial Sequence
  Primer
ccagtttttgtagatattaaagaaatcg

SEQ ID NO: 41 - DNA, 19 bp, Artificial Sequence
  Primer
ctgtagctgcaaaaaatcc

SEQ ID NO: 42 - DNA, 19 bp, Artificial Sequence
  Primer
atttggattttgcaattgc

SEQ ID NO: 43 - DNA, 24 bp, Artificial Sequence
  Primer
ttttcttgcgaaactgagccaacc

(19) United States
(12) Patent Application Publication
(10) Pub. No.: US 2004/0091935 A1
(43) Pub. Date: May 13, 2004

(54) Strains of Mycoplasma hyorhinis as causative agent for systemic sclerosis

(76) Inventor: Stephen J. Doxsey, Princeton, MA (US)

Correspondence Address: FISH & RICHARDSON PC, 225 FRANKLIN ST, BOSTON, MA, 02110, US

(21) Appl. No.: 10/391,249

(22) Filed: Mar. 17, 2003

Related U.S. Application Data
(60) Provisional application No. 60/364,801, filed on Mar. 15, 2002.

(51) Int. Cl. G01N 33/53

(52) U.S. Cl. 435/7.1

(57) ABSTRACT

The invention is based on the discovery that Mycoplasma hyorhinis (e.g., designated ATCC #PTA-3681) underlies the pathology of scleroderma and other autoimmune diseases. The invention encompasses Mycoplasma hyorhinis nucleic acids and polypeptides, methods of treating scleroderma and other autoimmune diseases by treating the underlying mycoplasma infection, compositions useful in treating scleroderma and other autoimmune diseases, methods of producing model organisms infected by mycoplasma, and a novel isolated strain of Mycoplasma hyorhinis designated ATCC #PTA-3681.

### TECHNICAL FIELD OF THE INVENTION

This invention relates to new strains of Mycoplasma hyorhinis, and the discovery that these new strains are causative agents for systemic sclerosis.

### BACKGROUND OF THE INVENTION

Systemic sclerosis (SSc)—also known as scleroderma—is a chronic autoimmune disease characterized by microvascular lesions, lymphocytic infiltrates, and fibrosis of the skin and visceral organs (Gavanescu et al., Journal of Clinical Immunology, 19(2):166-171, 1999). It afflicts women at four times the rate for men, and is relatively rare in children.

The Merck Manual of Diagnosis and Therapy, 17^{th} Edition (Beer and Berkow, Eds., The Merck Manual of Diagnosis and Therapy, 17^{th} Edition, 1999), for example, contains detailed information on the diagnosis, prognosis, and treatment of systemic sclerosis.

Systemic sclerosis exhibits a great range of variation in severity and progression (Beer and Berkow, Eds., The Merck Manual of Diagnosis and Therapy, 17^{th} Edition, 1999). At one extreme, it can manifest itself as generalized cutaneous thickening (systemic sclerosis with diffuse scleroderma), leading to rapid, progressive, and fatal visceral involvement. At another extreme, it can manifest itself as restricted skin involvement of the fingers and face, progressing slowly over the course of decades before internal involvement occurs.

The symptoms of systemic sclerosis vary from organ to organ and can be quite diverse (Beer and Berkow, Eds., The Merck Manual of Diagnosis and Therapy, 17^{th} Edition, 1999). Skin can become taut, shiny, and hyperpigmented, telangiectases can occur on the fingers, chest, face, lips, and tongue, and subcutaneous calcifications (known as calcinosis circumscripta) can develop on the fingertips and above bony eminences. The musculoskeletal system may suffer from such problems as friction rubs over joints, tendon sheaths, and large bursae due to deposition of fibrin on synovial surfaces. Most patients eventually experience some form of gastrointestinal tract esophageal dysfunction (e.g., dysphagia, gastroesophageal reflux disease). The cardiorespiratory system can suffer from such problems as lung fibrosis, pleurisy, pericarditis, pulmonary hypertension, cardiac arrhythmias, or heart failure. The renal system can suffer from severe renal disease due to intimal hyperplasia of interlobular and arcuate arteries.

No drug has significantly influenced the natural course of systemic sclerosis, but various drugs are of value in treating specific symptoms or organ systems (Beer and Berkow, Eds., The Merck Manual of Diagnosis and Therapy, 17^{th} Edition, 1999). These include corticosteroids, immunosuppressive drugs, antacids, and ACE inhibitors. For a more detailed account of systemic sclerosis, including its symptoms, signs, diagnosis, prognosis, and treatment, consult the Merck Manual of Diagnosis and Therapy (Beer and Berkow, Eds., The Merck Manual of Diagnosis and Therapy, 17^{th} Edition, 1999).

The majority of systemic sclerosis patients produce autoantibodies to proteins of intracellular organelles (Gavanescu et al., Journal of Clinical Immunology, 19(2):166-171, 1999). While many of these autoantibodies are shared among other autoimmune diseases, some are known to be specific for SSc and thus serve as useful immunological markers in disease diagnosis. The intracellular targets of SSc autoantibodies are found mainly in the nucleus. Among these are antibodies to DNA topoisomerase I and to centromere proteins on linetochores of chromosomes. While both classes of autoantibodies are good indicators of disease, only anti-topoisomerase autoantibodies are associated exclusively with SSc and not found in other autoimmune diseases. Another organelle that is a target of autoantibodies in SSc is the centrosome.

Centrosomes are intracellular organelles that nucleate microtubules and organize the bipolar mitotic spindle during cell division, although little is known of the molecular components involved in these functions (Gavanescu et al., Journal of Clinical Immunology, 19(2):166-171, 1999). By screening cDNA expression libraries using anti-centrosome autoantibodies from patients with SSc, two centrosome proteins have been discovered. To identify centrosome components, anti-centrosome autoantibodies from patients with SSc have been previously utilized to screen cDNA expression libraries. In this way pericentrin and CP140 (centrosome protein of 140 kDa) were discovered to be centrosome components. Using recombinant proteins from these cloned centrosome antigens and antibodies specific for these molecules, the prevalence of centrosome autoreactivity by immunofluorescence (IF) and Western blotting has been investigated.

Previous reports indicated that the prevalence of centrosome-specific autoantibodies in SSc was quite low (4-6%) compared to the prevalence of antinuclear antibodies (50-90%). It now appears that the reported centrosome reactivity was underestimated due to assay insensitivity and the small patient cohort examined (Gavanescu et al., Journal of Clinical Immunology, 19(2):166-171, 1999). The prevalence of anticenitrosome antibodies in scleroderma was reexamined using two novel assays for centrosome autoantigen detection. The first involves the use of two distinct recombinant centrosome proteins for probing patient sera by the Western blot technique. The second is an immunofluoresence (IF) assay that employs two unique modifications: the use of a centrosome-specific antibody to identify unequivocally centrosome reactivity of autoimmune sera in double-label IF reactions and a detergent prepermeabilization step that removes soluble cytoplasmic staining that often obscures the centrosome signal. As a consequence of these methodological optimizations, it has been shown that autoreactivity to the centrosome is more prevalent in SSc than reactivities to all other individual SSc autoantigens.

Phylogenetic studies suggest that all mycoplasmas have a monophyletic origin within the eubacteria. There are five recognized genera of mycoplasmas. One of these genera, the eponymous genus Mycoplasma, contains the vast majority of recognized species. The Mycoplasma genus currently includes of more than 60 known species that are differentiated on the basis of various tests, including utilization of glucose and mannose, arginine hydrolysis, phosphatase production, the “film and spots” reaction, and haemadsorption.

Mycoplasma is a genus of sterol-requiring, catalase-negative pathogens commonly found in the respiratory and urogenital tracts of humans and other animals. The cells of Mycoplasma are typically non-motile and pleomorphic, ranging from spherical, ovoid or pear-shaped to branched filamentous forms. Filaments are the typical forms in young cultures under optimal conditions, which subsequently transform into chains of coccoid cells that later break up into individual cells that are capable of passing through membrane filters of pore size 0.45 μm, or even 0.22 μm. A trilaminar cytoplasmic membrane contains sterols, phospholipid and proteins. Therefore, the cells are generally susceptible to polyene antibiotics and to lysis by digitonin. Replication of the Mycoplasma genome may precede cytoplasmic division resulting in multinucleate filaments before individual cells are delimited by constriction. Budding can also occur.

### SUMMARY OF THE INVENTION

The invention is based on the discovery and isolation of new strains of mycoplasma, including murine and human strains of Mycoplasma hyorhinis designated ATCC #PTA-3681, that can infect host organisms, causing systemic sclerosis. The invention provides new methods of treating systemic sclerosis, as well as other autoimmune diseases, by inhibiting or preventing underlying mycoplasma infection.

The invention encompasses isolated nucleic acid sequences, including SEQ ID NOS:1-22. The invention also encompasses isolated polypeptides encoded by SEQ ID NOS:1-22. These nucleic acids or polypeptides can consist only of SEQ ID NOs: 1-22 and polypeptides encoded by SEQ ID NOs:1-22, respectively, or they can be included as part of larger sequences.

The invention features an isolated Mycoplasma hyorhinis (e.g., designated ATCC #PTA-3681). It also features an isolated tissue culture that includes Mycoplasma hyorhinis (e.g., designated ATCC #PTA-3681).

Also featured in the invention are mammalian models (e.g., mouse, pig, or other mammal) of autoimmune disease (e.g., systemic sclerosis) in which the mammals are infected with a sufficient amount of isolated Mycoplasma hyorhinis (e.g., designated ATCC #PTA-368 1) to produce symptoms of autoimmune disease.

The invention encompasses methods of producing a mammalian model (e.g., mouse, pig, or other mammal) of systemic sclerosis in which the method includes exposing, a mammal to an amount of isolated Mycoplasma hyorhinis (e.g., designated ATCC #PTA-3681) sufficient to induce infection, in which the infection induces symptoms of systemic sclerosis.

Also encompassed by the invention are methods of treating an animal (e.g., a mammal (e.g., mouse, rat, dog, cat, pig, sheep, goat, cow, horse, monkey, chimpanzee, or human), or a bird (e.g., chicken, duck, goose, turkey, pheasant, pigeon, parrot, toucan, budgie, or canary)) having symptoms of systemic sclerosis, in which the method includes: obtaining a sample from the animal; testing the sample for the presence of Mycoplasma hyorhinis (e.g., designated ATCC #PTA-3681); and (e.g., if the sample contains the Mycoplasma hyorhinis), administering to the animal an amount of a composition (e.g., including an antibody (e.g., monoclonal) or aptamer that specifically binds to Mycoplasma hyorhinis, an antisense nucleic acid that specifically hybridizes to an mRNA complementary to any one of SEQ ID NOs:1-22, an ribozyme that specifically cleaves an mRNA transcript of any one of SEQ ID NOs:1-22, an siRNA or dsRNA, an antibiotic (e.g., chloramphenicol, tetracycline, ciprofloxacin, or doxicycline), a vaccine (e.g., a DNA vaccine)) effective to treat the Mycoplasma hyorhinis within the animal, thereby reducing the severity of the systemic sclerosis.

The invention also includes methods for treating systemic sclerosis in a subject in which the method includes: providing an organism, tissue, or cells infected with Mycoplasma hyorhinis (e.g., designated ATCC #PTA-3681); and administering to the subject or the subject's tissue an amount of a composition effective to inhibit, reduce, or eliminate the Mycoplasma hyorhinis within the animal, thereby reducing the severity of the systemic sclerosis.

The invention also features methods of detecting Mycoplasma hyorhinis (e.g., designated ATCC #PTA-3681) in a sample, in which the method includes obtaining a sample and determining whether any nucleic acid sequence in the sample is identical to a nucleotide sequence of SEQ ID NOs:1-22, in which sequence identity indicates the presence of Mycoplasma hyorhinis in the sample.

The invention further features methods of detecting Mycoplasma hyorhinis (e.g., designated ATCC #PTA-3681) in a sample, in which the method includes obtaining a sample from an organism, and detecting in the sample the presence of antibodies that specifically bind to Mycoplasma hyorhinis, in which the presence of the antibodies indicates the presence of Mycoplasma hyorhinis in the sample.

The invention also encompasses isolated antibodies that specifically bind to Mycoplasma hyorhinis (e.g., designated ATCC #PTA-3681).

The invention also features isolated aptamers that specifically bind to Mycoplasma hyorhinis (e.g., designated ATCC #PTA-3681).

The invention further encompasses methods for detecting in a biological sample an antibody that specifically binds to an antigenic site of a Mycoplasma hyorhinis (eg., designated ATCC #PTA-368 1), in which the method includes contacting the sample with an antigen isolated (e.g., by sonication or detergent extraction) from a Mycoplasma hyorhinis, and measuring the formation of an antigen-antibody complex, in which the presence of a complex indicates the presence of the antibody.

In addition, the invention features methods for diagnosing systemic sclerosis in a subject, in which the method includes obtaining a biological sample (e.g., skin tissue, such as lesioned skin tissue) from the subject, contacting the sample with antigen of Mycoplasma hyorhinis (e.g., designated ATCC #PTA-3681), and measuring the formation of antigen-antibody complex, wherein the presence of a complex indicates that the subject has systemic sclerosis.

The invention also features a composition that includes purified antigen from Mycoplasma hyorhinis (e.g., designated ATCC #PTA-3681).

The invention also encompasses methods for treating systemic sclerosis, in which the method includes the steps of providing an organism, tissue, or cells infected with Mycoplasma hyorhinis (e.g., designated ATCC #PTA-3681), and applying antibiotics to said tissues in a dosage effective to lower the amount of the Mycoplasma hyorhinis designated ATCC #PTA-3681 to an amount sufficiently low to reduce or eliminate systemic sclerosis.

In addition, the invention encompasses a method of inducing centrosome autoantibodies in a cell by infecting the cell with Mycoplasma hyorhinis designated ATCC #PTA-3681.

The invention also features a method of generating antibodies specific for Mycoplasma hyorhinis (e.g., designated ATCC #PTA-3681), in which the method includes providing antigen from Mycoplasma hyorhinis, and introducing into an animal (e.g., a mammal or a bird) a composition that includes antigen from Mycoplasma hyorhinis, in which antibodies specific for Mycoplasma hyorhinis are generated.

The invention further encompasses methods for treating a bird having a mycoplasma infection, in which the method includes obtaining a sample from the bird, testing the sample or samples for the presence of mycoplasma; and, if the sample contains the mycoplasma, administering to the infected bird an amount of a composition effective to inhibit, reduce, or eliminate the mycoplasma within the bird, thereby reducing the severity of the infection.

The invention also includes permutations and combinations of the features of the methods and compositions described above.

As used herein, the term “treat” means to inhibit, reduce, or eliminate symptoms or underlying causes of a disease or disorder.

As used herein, the terms “systemic sclerosis” and “scleroderma” are synonyms.

As used herein, the phrase “Koch's postulates” refers to observation that, to establish the specificity of a pathogenic microorganism, it must be present in all cases of the disease, inoculations of its pure cultures must produce disease in animals, and from these it must be again obtained and be propagated in pure cultures.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention provides several advantages. Among them are new methods of treating scleroderma and other autoimmune diseases, by inhibiting the underlying mycoplasma infection, a novel strain of Mycoplasma hyorhinis that can be used to study scleroderma (including its causation and treatment), and a means of identifying and inhibiting the pathogens that cause autoimmune diseases generally.

Other features and advantages of the invention will be apparent from the following detailed description, and from the claims.

### BRIEF DESCRIPTION OF THE DRAWINGS

FIGS. 1A and 1B are electron micrographs depicting the ultrastructure of Mycoplasma hyorhinis both during cell infection (FIG. 1A) and in fractions (FIG. 1B).

FIG. 2 is a representation of Mycoplasma hyorhinis PDH (pigment dispersing hormone) DNA sequences from pig and mouse tissue (SEQ ID NO:1 -3). The consensus sequence is darkened.

FIG. 3 is a representation of Mycoplasma hyorhinis perM (permease) DNA sequences from pig and mouse tissue (SEQ ID NO:4-6). The consensus sequence is darkened.

FIG. 4 is a representation of Mycoplasma hyorhinis MET (methylase) DNA sequences from pig and mouse tissue (SEQ ID NO:7-9). The consensus sequence is darkened.

FIG. 5 is a representation of Mycoplasma hyorhinis TPI (triose phosphate isomerase) DNA sequences from pig, mouse, and human tissue (SEQ ID NO:10-12). The consensus sequence is darkened.

FIG. 6 is a representation of Mycoplasma hyorhinis 16S (16S ribosomal RNA) DNA sequence from mouse tissue (SEQ ID NO:13). The consensus sequence is darkened.

FIG. 7 is a representation of Mycoplasma hyorhinis VLP (variable lipoprotein) DNA sequences from mouse tissue (SEQ ID NO:14-15). The consensus sequence is darkened.

FIG. 8 is a representation of Mycoplasma hyorhinis 16S (16S ribosomal RNA) DNA sequences from pig, mouse, and human tissue (SEQ ID NO:16-18). The consensus sequence is darkened.

FIG. 9 is a representation of Mycoplasma hyorhinis VLP (variable lipoprotein) DNA sequence from mouse tissue (SEQ ID NO:19). The consensus sequence is darkened.

FIG. 10 is a representation of Mycoplasma hyorhinis TUF (elongation factor tu) DNA sequences from pig, mouse, and human tissue (SEQ ID NO:20-22). The consensus sequence is darkened.

FIG. 11 is a representation of immunoblots showing that strong autoreactivity develops in mice at two weeks to the centrosome proteins pericentrin and centriolin in mice. a=mouse a. b=mouse b. Pc=pericentrin. Cen=centriolin.

FIG. 12 is a graph showing inhibition of Mycoplasma hyorhinis by antibiotics. Each point on the graph is an average of two experiments. Inf.=infection agent. Dox=doxicyclin. Cip=ciprofloxacin.

FIG. 13 is a graph showing the banding pattern of an infectious agent (Mycoplasma hyorhinis) from culture supernatants on a renografin density gradient. Pfu is indicated by diamonds. Refractive index is indicated by squares.

FIGS. 14A and B are graphs of anti-centrosome IgG and IgM titers, respectively. Titers were determined by endpoint titration using the immunofluorescence assay and are expressed as the reciprocal of the serum dilution. Each point represents the result for an individual mouse. Results are representative of 4 independent experiments. Horizontal bars represent mean of all values.

FIG. 15 is a representations of immunoblots of recombinant pericentrin showing two dilutions (1/60 and 1/300) of serum samples from mice infected orally (lanes 1-8), intranasally (lanes 9-16), intravenously (lanes 17-24), or intraperitoneally (lanes 25-32). Results are representative of three separate experiments.

FIGS. 16A and B are graphs of autoantibody titers in control mice and in mice treated with the bacteriostatic antibiotic ciprofloxacin pre-infection (FIG. 16A) and six weeks post-infection (FIG. 16B). Cip=ciprofloxacin. Myc=mycoplasma. Each point represents data from a single mouse. Horizontal bars represent mean values of all points.

FIGS. 17A and B are a pair of graphs of autoantibody titers in ER-associated and centrosome autoantibodies in Mycoplasma-infected mice for both Pan-specific (FIG. 17A) and IgM-specific (FIG. 17B) Igs. Myc=mycoplasma. C=centrosome. ER=endoplasmic reticulum.

Like reference symbols in the various drawings indicate like elements.

### DETAILED DESCRIPTION OF THE INVENTION

Systemic sclerosis, also known as scleroderma, typically involves the development of autoantibodies to intracellular proteins, a feature shared with other autoimmune diseases. A significant number of scleroderma patients develop autoantibodies to intracellular organelles called centrosomes. In murine experimental systems uninfected mice develop autoantibodies to centrosome proteins after exposure to mice that have already developed such autoantibodies, demonstrating the presence of an infectious agent. The agent was isolated vitro by its ability to plaque on cultured cell monolayers, and was characterized and identified as a murine homologue of Mycoplasma hyorhinis. When reintroduced into naive wild-type mice, plaque-purified murine Mycoplasma triggered the development of anti-centrosome autoantibodies, thus fulfilling Koch's postulates. Moreover, DNA sequences homologous to M. hyorhinis DNA were amplified from skin lesions of human scleroderma patients, but not from patients with skin lesions that lacked an autoimmune component Such as basal cell carcinoma. These findings indicate that some Mycoplasma, may be a cofactor in the pathogenesis of human autoimmune diseases, as well as autoimmune diseases in other mammals (e.g., mouse, rat, pig, goat, sheep, cow).

Mycoplasma are also known to cause infections in birds (e.g, poultry, such as chickens, turkeys, ducks, and geese). The compositions and methods of the invention can also be adapted to treatment and prevention of mycoplasma infection and diseases (e.g., eye diseases) of birds caused by mycoplasma.

### Mycoplasmas

Mycoplasmas are a group of single-celled prokaryotic microorganisms that have the smallest known cells and are the smallest known self-replicating organisms. They are tiny organisms that can be as small as 0.1 μm in diameter. A mycoplasma cell is also notable for lacking a cell wall. Each cell consists of an external plasma membrane, DNA, RNA, ribosomes, soluble proteins, sugars, and lipids. The genetic material is minimal, and probably contains fewer than 650 genes. By comparison, Escherichia coli, and other common eubacteria, typically contain at least five times as many genes.

Phylogenetic studies suggest that all mycoplasmas have a monophyletic origin within the eubacteria. There are five recognized genera of mycoplasmas. One of these genera, the eponymous genus Mycoplasma, contains the vast majority of recognized species. The Mycoplasma genus currently consists of more than 60 known species that are differentiated on the basis of various tests, including utilization of glucose and mannose, arginine hydrolysis, phosphatase production, the “film and spots” reaction, and haemadsorption.

Mycoplasma is a genus of sterol-requiring, catalase-negative pathogens commonly found in the respiratory and urogenital tracts of humans and other animals. The cells of Mycoplasma are typically non-motile and pleomorphic, ranging from spherical, ovoid or pear-shaped to branched filamentous forms. Filaments are the typical forms in young cultures under optimal conditions, which subsequently transform into chains of coccoid cells which later break up into individual cells that are capable of passing through membrane filters of pore size 0.45 μm, or even 0.22 μm. A trilaminar cytoplasmic membrane contains sterols, phospholipid and proteins. Therefore, the cells are generally susceptible to polyene antibiotics and to lysis by digitonin. Replication of the Mycoplasma genome may precede cytoplasmic division resulting in multinucleate filaments before individual cells are delimited by constriction. Budding can also occur.

Mycoplasmas are not obligatory intracellular microorganisms and are usually found extracellularly, but can be found intracellularly in infected tissues (Wolfgang et al. Eds., “Mycoplasma”, In Microbiology, 19^{th} Edition, pgs. 617-623, 1988).

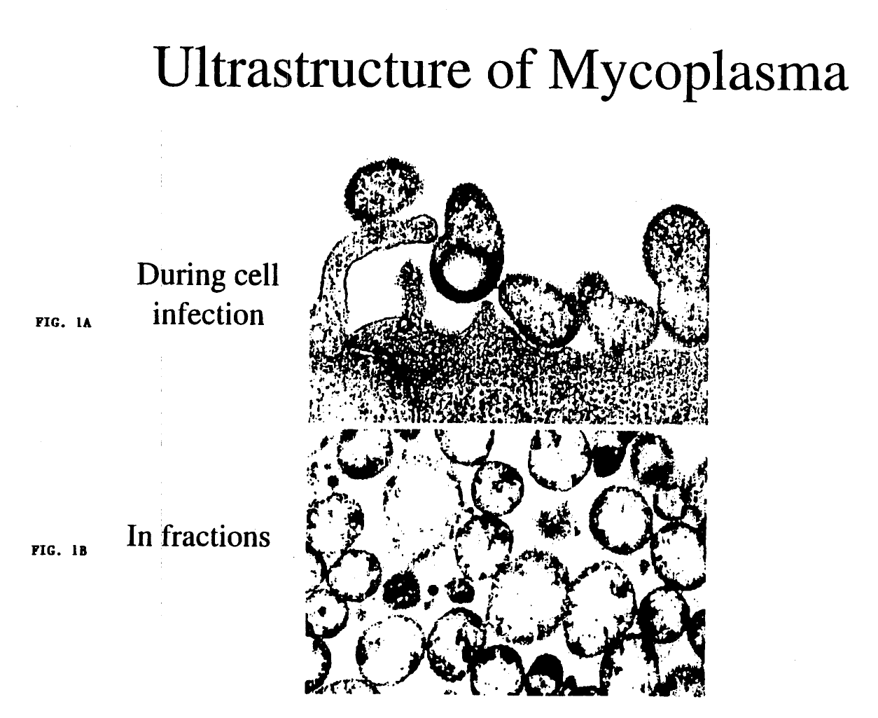

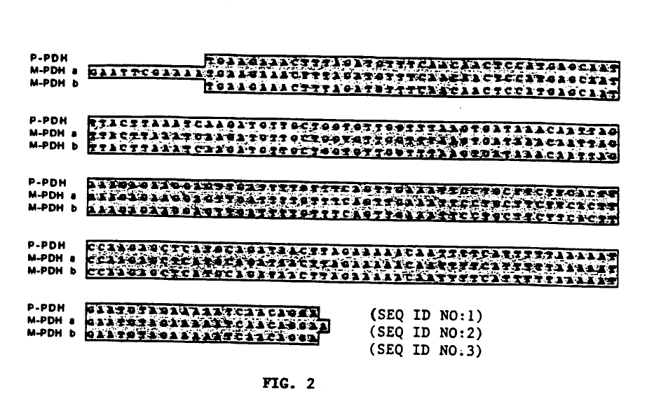

Even today with electron microscopy, it is still often difficult to differentiate the mycoplasmas from the cellular protoplasmic processes or the subcellular organelles of the infected host, because ultrastructurally, these microorganisms have protoplasm-like internal structures and are bounded by only an outer limited membrane (unit membrane) without a cell wall. Thus, there have been few electron microscopic studies of mycoplasmas identified directly in the infected tissues of animals or humans. FIG. 1 depicts Mycoplasma hyorhinis both during infection (FIG. 1A) and in fractions (FIG. 1B).

It has been reported that ultrastructural examination of infected tissues has failed to localize the microbe, even in tissues where very high titers (>10⁹/gm) of microorganisms were recovered in culture (Elizan et al., Pro. Soc. Exp. Biol. Med., 139:52, 1972; Schwartz et al., Pro. Soc. Exp. Biol. Med., 139:56, 1972). Therefore, morphologically, the microbe might be mimicking certain normal cellular or subcellular structures in the infected host tissues and preventing direct visual identification.

In addition to the natural difficulty of morphological differentiation between the microorganisms and the protoplasm of infected cells, the often poorly preserved formalin-fixed clinical materials present further limitations to any attempt to directly visualize mycoplasma organisms in the tissues.

### Autoantibodies and Autoimmune Disease

The mechanism of autoantibody production in autoimmune diseases and their role in pathogenesis is largely unknown. A number of mechanisms for generating autoantibodies in these diseases have been proposed. These include generation of antibodies against epitopes of pathogens that share homology to cellular proteins (molecular mimicry), generation of atypical antigenic epitopes by pathogen protease-mediated cleavage of cellular protein and loss of immune tolerance. However, a direct relationship has not been established between any of these proposed mechanisms and autoantibody production.

Systemic sclerosis, also known as SSc or scleroderma, is an autoimmune disease of unknown etiology, characterized by vascular lesions, aberrant deposition of collagen in the skin and visceral organs, chronic inflammation and fibrosis of the skin and other organs, and systemic production of autoantibodies to intracellular structures. Individuals with scleroderma produce antibodies against centrosomes and other intracellular organelles. Centrosomes are cellular organelles involved in mitotic spindle organization and cell cycle progression. They are major targets of autoantibodies in the autoimmune disease scleroderma and in other autoimmune diseases to a lesser extent (Gavanescu et al., Journal of Clinical Immunology, 19(2):166-171, 1999).

### Mouse Model of Systemic Sclerosis

The invention includes the use of M. hyorhinis, which can induce the production of centrosome autoantibodies in mice, to produce a model organism useful for studying mechanisms of autoantibody production. The production of model mice infected by M. hyorhinis can be facilitated by relying on either of two sources of pre-infected starting mice: (1) production of mutant mice that lack certain immune components; or (2) use of currently available strains of mice.

Various strains of mice (e.g., Balb.c/SvJ, C57BL/6J and 129/A) can be used to induce centrosome autoantibodies by introducing murine mycoplasma hyorhinis intransally. These mice provide a valuable model for testing the role of various immune components in this process. For example mice mutant for T-cells could be tested for their ability to mount a mycoplasma-induced autoimmune response.

### New Mouse and Human Strains of Mycoplasma hyorhinis

The invention encompasses new murine and human forms of M. hyorhinis. These strains are different from porcine M. hyorhinis, as well as from each other, suggesting that they represent species-specific forms. It is also possible that they represent porcine forms that have mutated after living in the mouse and human. Because the M. hyorhinis strains of the invention are different from those found in pigs, they can be referred to as murine M. hyorhinis and human H. hyorhinis.

### Nucleic Acids

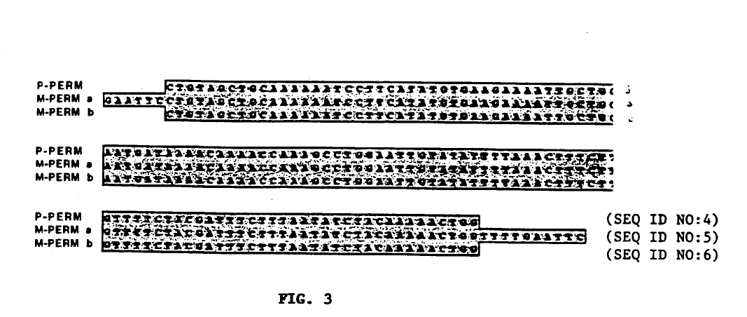

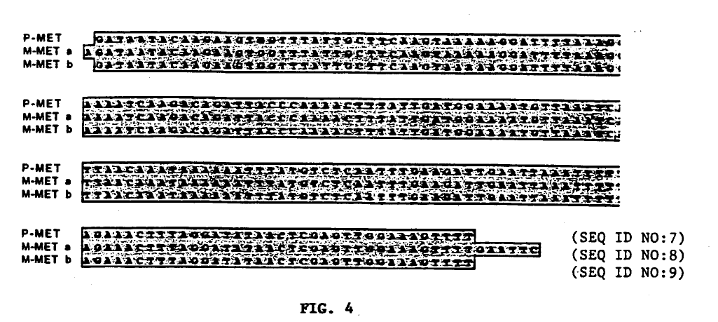

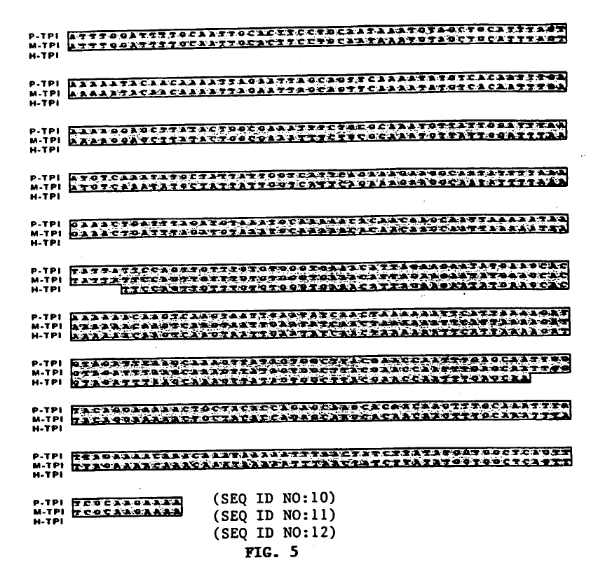

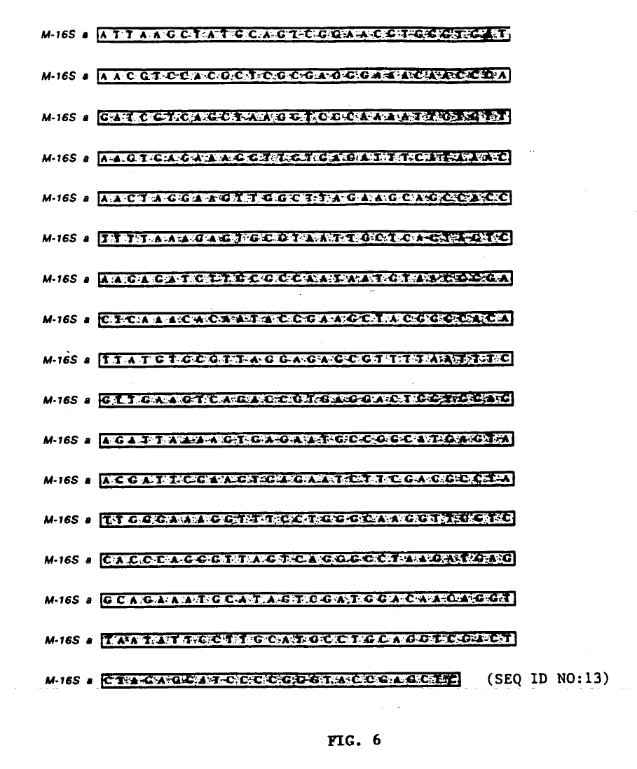

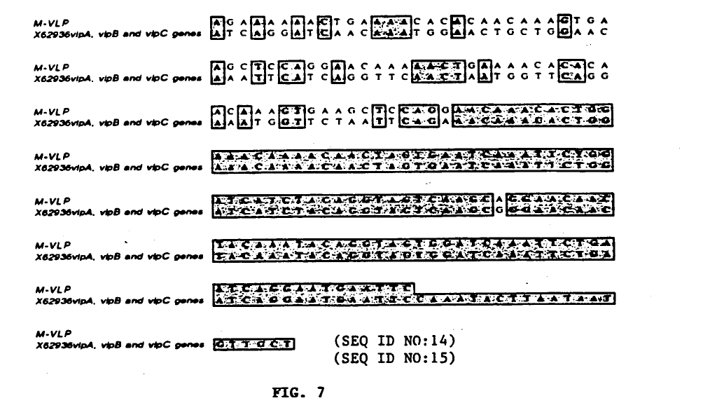

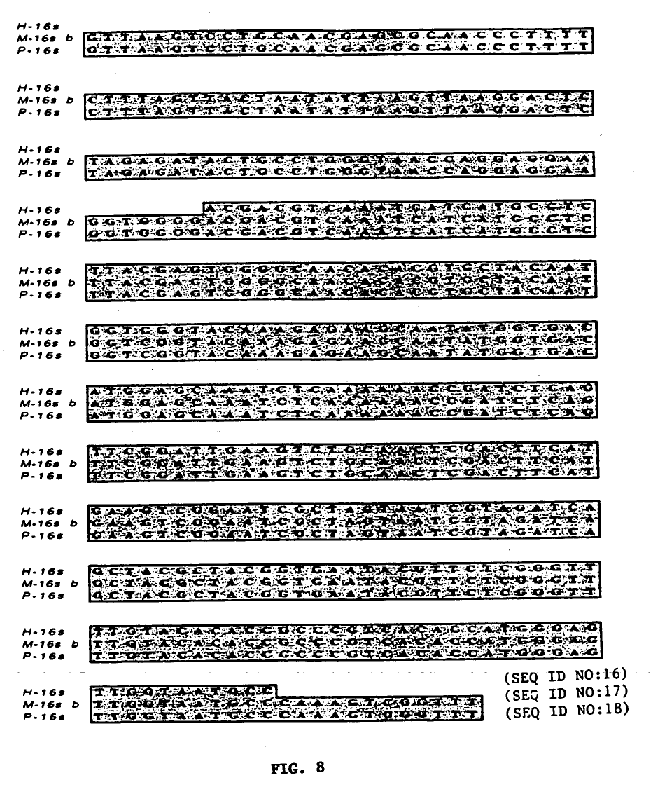

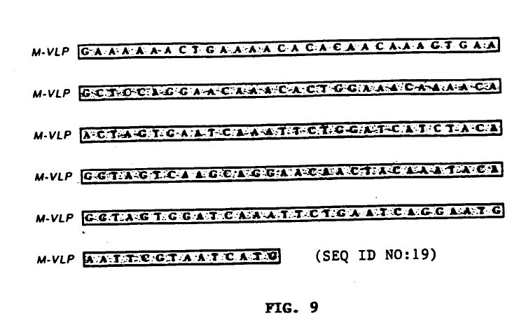

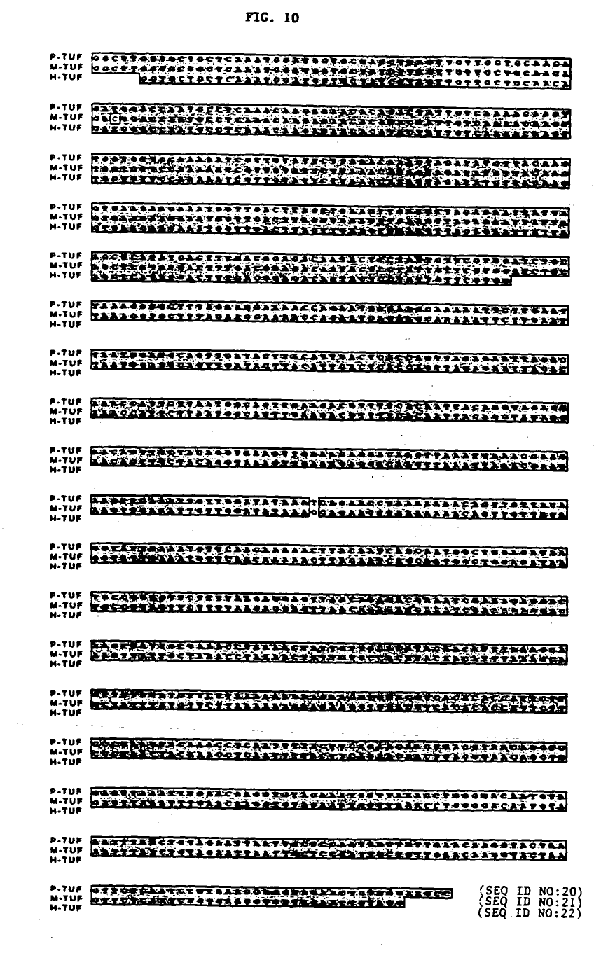

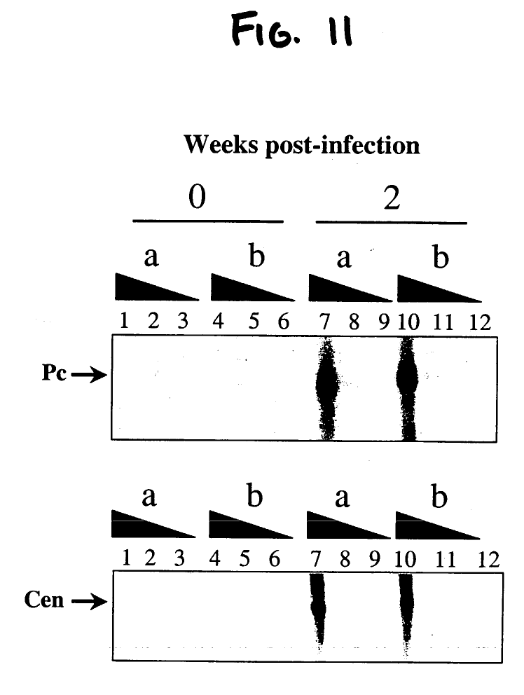

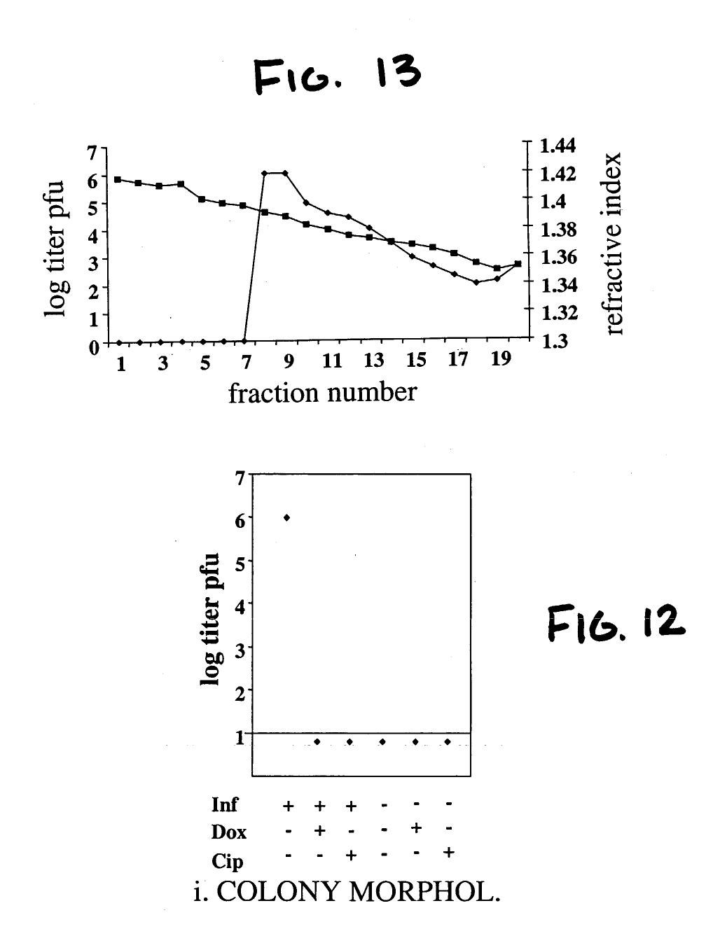

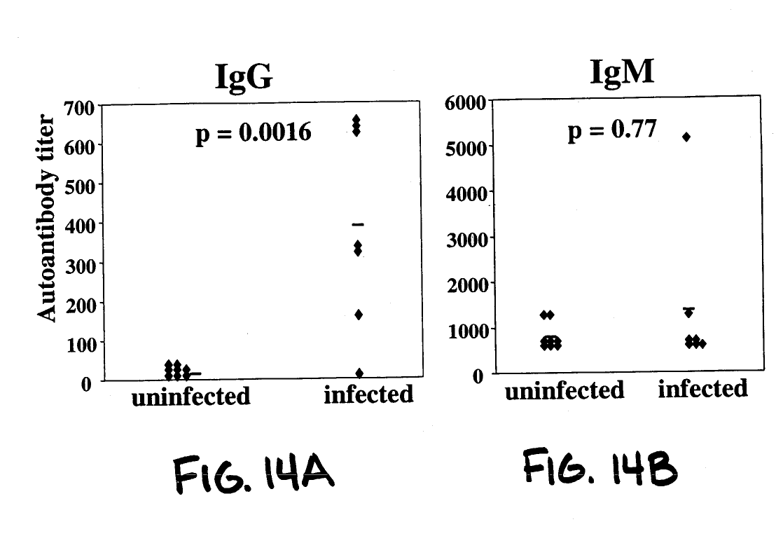

The invention encompasses nucleic acids that have sequences substantially identical to any one of the nucleic acid sequences of SEQ ID NO:1-22, which are shown in FIGS. 2-10. A nucleic acid sequence that is substantially identical to a given reference nucleic acid sequence is a nucleic acid having a sequence that has at least 85% identity to the sequence of the given reference nucleic acid sequence, e.g., the nucleic acid sequence of SEQ ID NO:2. Of course, a substantially identical sequence can have a greater percentage of identity, e.g., 90%, 95%, 96%, or 99% identity.

The nucleic acid molecules of the invention can be cDNA, genomic DNA, synthetic DNA, or RNA, and can be double-stranded or single-stranded (i.e., either a sense or all antisense strand). Fragments of these molecules are also considered within the scope of the invention, and can be produced, for example, by the polymerase chain reaction (PCR) or generated by treatment with one or more restriction endonucleases. A ribonucleic acid (RNA) molecule can be produced by in vitro transcription.

The nucleic acid molecules of the invention can contain naturally occurring sequences, or sequences that differ from those that occur naturally, but, due to the degeneracy of the genetic code, encode the same polypeptide. In addition, these nucleic acid molecules are not limited to sequences that only encode polypeptides, and thus, can include some or all of the non-coding sequences that lie upstream or downstream from a coding sequence.

The nucleic acid molecules of the invention can be synthesized (for example, by phosphoramidite-based synthesis) or obtained from a biological cell, such as a M. hyorhinis cell. Combinations or modifications of the nucleotides within these types of nucleic acids are also encompassed.

In addition, the isolated nucleic acid molecules of the invention encompass fragments that are not found as such in the natural state, but possess the same, or essentially the same, functions or uses as the full length nucleic acids. The lengths of such fragments can range from very short to almost as long as the full length nucleic acids of which they are fragments. For example, fragments of SEQ ID NO:2 can range in length from 10 to 210 nucleic acids in length (e.g., 10, 20, 30, 40, 50, 60, 70, 80, 90, 100, 110, 120, 130, 140, 150, 160, 170, 180, 190, 200, or 210 nucleic acids). Thus, the invention encompasses recombinant molecules, such as those in which a nucleic acid molecule (for example, an isolated nucleic acid molecule such as SEQ ID NO:2) is incorporated into a vector (for example, a plasmid or viral vector) or into the genome of a heterologous cell (or the genome of a homologous cell, at a position other than the natural chromosomal location). Recombinant nucleic acid molecules and uses therefor are discussed further below.

In the event the nucleic acid molecules of the invention encode or act as antisense molecules, they can be used for example, to regulate translation of mRNA of the invention. Techniques associated with detection or regulation of expression of nucleic acids or polypeptides of the invention are well known to skilled artisans and can be used to diagnose and/or treat disorders associated with aberrant expression of nucleic acids or polypeptides of the invention.

The invention also encompasses nucleic acid molecules that hybridize under stringent conditions to a nucleic acid molecule encoding a polypeptide of the invention. The cDNA sequences described herein can be used to identify these hybridizing nucleic acids, which include, for example, nucleic acids that encode homologous polypeptides in other species, and splice variants of the genes of the invention. Accordingly, the invention features methods of detecting and isolating these nucleic acid molecules. Using these methods, a sample (for example, a nucleic acid library, such as a cDNA or genomic library) is contacted (or “screened”) with a probe specific to a nucleotide of the invention (for example, a fragment of SEQ ID NO:2 that is at least 25 or 50 or 100 nucleotides long). The probe will selectively hybridize to nucleic acids encoding related polypeptides (or to complementary sequences thereof). The probe, which can contain at least 25 (for example, 25, 50, 100, 200, 300, or more than 300 nucleotides) can be produced using any of several standard methods (see, for example, Austubel et al., “Current Protocols in Molecular Biology, Vol. 1,” Green Publishing Associates, Inc., and John Wiley & Sons, Inc., New York, 1989). For example, the probe can be generated using PCR amplification methods in which oligonticleotide primers are used to amplify a nucleic acid sequence specific to a nucleic acid of the invention that can be used as a probe to screen a nucleic acid library and thereby detect nucleic acid molecules (within the library) that hybridize to the probe.

One single-stranded nucleic acid is said to hybridize to another if a duplex forms between them. This occurs when one nucleic acid contains a sequence that is the reverse and compliment of the other (this same arrangement gives rise to the natural interaction between the sense and antisense strands of DNA in the genome and underlies the configuration of the “double helix”). Complete complementarity between the hybridizing regions is not required for a duplex to fore; it is only necessary that the number of paired bases is sufficient to maintain the duplex under the hybridization conditions used.

Typically, hybridization conditions are of low to moderate stringency. These conditions favor specific interactions between completely complementary sequences, but allow some non-specific interaction between less than perfectly matched sequences to occur as well. After hybridization, the nucleic acids can be “washed” under moderate or high conditions of stringency to dissociate duplexes that are bound together by some non-specific interaction (the nucleic acids that form these duplexes are thus not completely complementary).

As is known in the art, the optimal conditions for washing are determined empirically, often by gradually increasing the stringency. The parameters that can be changed to affect stringency include, primarily, temperature and salt concentration. In general, the lower the salt concentration and the higher the temperature, the higher the stringency. Washing can be initiated at a low temperature (for example, room temperature) using a solution containing a salt concentration that is equivalent to or lower than that of the hybridization solution. Subsequent washing can be carried out using progressively warmer solutions having the same salt concentration. As alternatives, the salt concentration can be lowered and the temperature maintained in the washing step, or the salt concentration can be lowered and the temperature increased. Additional parameters can also be altered. For example, use of a destabilizing agent, such as formamide, alters the stringency conditions.

In reactions where nucleic acids are hybridized, the conditions used to achieve a given level of stringency will vary. There is not one set of conditions, for example, that will allow duplexes to form between all nucleic acids that are 85% identical to one another; hybridization also depends on unique features of each nucleic acid. The length of the sequence, the composition of the sequence (for example, the content of purine-like nucleotides versus the content of pyrimidine-like nucleotides) and the type of nucleic acid (for example, DNA or RNA) affect hybridization. An additional consideration is whether one of the nucleic acids is immobilized (for example, on a filter).

An example of a progression from lower to higher stringency conditions is the following, where the salt content is given as the relative abundance of SSC (a salt solution containing sodium chloride and sodium citrate; 2×SSC is 10-fold more concentrated than 0.2×SSC). Nucleic acids are hybridized at 42° C. in 2×SSC/0.1% SDS (sodium dodecylsulfate; a detergent) and then washed in 0.2×SSC/0.1% SDS at room temperature (for conditions of low stringency); 0.2×SSC/0.1% SDS at 42° C. (for conditions of moderate stringency); and 0.1×SSC at 68° C. (for conditions of high stringency). Washing can be carried out using only one of the conditions given, or each of the conditions can be used (for example, washing for 10-15 minutes each in the order listed above). Any or all of the washes can be repeated. As mentioned above, optimal conditions will vary and can be determined empirically.

Once detected, the nucleic acid molecules can be isolated by any of a number of standard techniques (see, for example, Sambrook et al., “Molecular Cloning, A Laboratory Manual,” 2nd Ed. Cold Spring Harbor Laboratory Press, Cold Spring Harbor, N.Y., 1989).

The invention also encompasses: (a) expression vectors that contain any of the foregoing coding sequences (related to a polypeptide of the invention) and/or their complements (that is, “antisense” sequence); (b) expression vectors that contain any of the foregoing coding sequences (related to a polypeptide of the invention) operatively associated with a regulatory element (examples of which are given below) that directs the expression of the coding sequences; (c) expression vectors containing, in addition to sequences encoding a polypeptide of the invention, nucleic acid sequences that are unrelated to nucleic acid sequences encoding a polypeptide of the invention, such as molecules encoding a reporter or marker; and (d) genetically engineered host cells that contain any of the foregoing expression vectors and thereby express the nucleic acid molecules of the invention in the host cell.

The invention also encompasses heterologous fusions with endogenous human and murine genes. For example, many human oncogenes are hybrids comprised of a viral domain and a human domain. This suggests that, at some point, a virus integrated its sequence into a human, thus creating a chimeric sequence that is oncogenic. An analogous phenomenon can occur involving mycoplasmas.

Recombinant nucleic acid molecules can contain a sequence encoding a soluble polypeptide of the invention; mature polypeptide of the invention; or polypeptide of the invention having an added or endogenous signal sequence. A full-length polypeptide of the invention; a domain of a polypeptide of the invention; or a fragment thereof may be fused to additional polypeptides, as described below. Similarly, the nucleic acid molecules of the invention can encode the mature form of a polypeptide of the invention or a form that encodes a polypeptide that facilitates secretion. In the latter instance, the polypeptide is typically referred to as a proprotein (or preprotein), which can be converted into an active form by removal of the signal sequence, for example, within the host cell. Proproteins can be converted into the active form of the protein by removal of the inactivating sequence.

The regulatory elements referred to above include, but are not limited to, inducible and non-inducible promoters, enhancers, operators and other elements, which are known to those skilled in the art, and which drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Similarly, the nucleic acid can form part of a hybrid gene encoding additional polypeptide sequences, for example, sequences that function as a marker or reporter. Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter. Generally, the hybrid polypeptide will include a first portion and a second portion; the first portion being a polypeptide of the invention and the second portion being, for example, the reporter described above or an immunoglobulin constant region.

The expression systems that may be used for purposes of the invention include, but are not limited to, microorganisms such as bacteria (for example, E. coli and Bacillis subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecules of the invention (preferably containing the nucleic acid sequence encoding a polypeptide of the invention); insect cell systems infected with recombinant virus expression vectors (for example, baculovirus) containing the nucleic acid molecules of the invention; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing nucleotide sequences of nucleic acids of the invention; or mammalian cell systems (for example, COS, CHO, BHK, 293, VERO, HeLa, MDCK, WJ38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors may be advantageously selected depending upon the use intended for the gene product being expressed. For example, when a large quantity of such a protein is to be produced, for example, for the generation of pharmaceutical compositions containing polypeptides of the invention or for raising antibodies to those polypeptides, vectors that are capable of directing the expression of high levels of fusion protein products that are readily purified may be desirable. Such vectors include, but are not limited to, the E. coli expression vector pUR278 (Ruther et al., EMBO J. 2:1791, 1983), in which the coding sequence of the insert may be ligated individually into the vector in frame with the lacZ coding region so that a fusion protein is produced; pIN vectors (Inouye and Inouye, Nucleic Acids Res., 13:3101-3109, 1985; Van Heeke and Schuster, J. Biol. Chem., 264:5503-5509, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) can be used as a vector to express foreign genes. The virus grows in Spodoptera frugiperda cells. The coding sequence of the insert may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant vinises are then used to infect Spodoptera frugiperda cells in which the inserted gene is expressed. (for example, see Smith et al., J. Virol., 46:584, 1983; Smith, U.S. Pat. No. 4,215,051).

In mammalian host cells, a number of viral-based expression systems may be utilized. In cases where an adenovirus is used as an expression vector, the nucleic acid molecule of the invention may be ligated to an adenovirus transcription/translation control complex, for example, the late promoter and tripartite leader sequence. This chimeric gene may then be inserted in the adenovirus genome by in vitro or in vivo recombination. Insertion in a non-essential region of the viral genome (for example, region E1 or E3) will result in a recombinant virus that is viable and capable of expressing a gene product of the invention in infected hosts (for example, see Logan and Shenk, Proc. Natl. Acad. Sci. USA, 81:3655-3659, 1984). Specific initiation signals may also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol., 153:516-544, 1987).

In addition, a host cell strain may be chosen which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. The mammalian cell types listed above are among those that could serve as suitable host cells.

For long-term, high-yield production of recombinant proteins, stable expression is preferred. For example, cell lines that stably express the sequences of nucleic acids or polypeptides of the invention described above may be engineered. Rather than using expression vectors that contain viral origins of replication, host cells can be transformed with DNA controlled by appropriate expression control elements (for example, promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci that in turn can be cloned and expanded into cell lines. This method can advantageously be used to engineer cell lines that express nucleic acids or polypeptides of the invention. Such engineered cell lines may be particularly useful in screening and evaluation of compounds that affect the endogenous activity of the gene product.

A number of selection systems can be used. For example, the herpes simplex virus thymidine kinase (Wigler, et al., Cell, 11:223, 1977), hypoxanthine-guanine phosphoribosyltransferase (Szybalska and Szybalski, Proc. Natl. Acad. Sci. USA, 48:2026, 1962), and adenine phosphoribosyltransferase (Lowy et al., Cell, 22:817, 1980) genes can be employed in tk⁻, hgprt⁻ or aprt⁻ cells, respectively. Also, anti-metabolite resistance can be used as the basis of selection for the following genes: dhfr, which confers resistance to methotrexate (Wigler et al., Proc. Natl. Acad. Sci. USA, 77:3567, 1980; O'Hare et al., Proc. Natl. Acad. Sci. USA, 78:1527, 1981); gpt, which confers resistance to mycophenolic acid (Mulligan and Berg, Proc. Natl. Acad. Sci. USA, 78:2072, 1981); neo, which confers resistance to the aminoglycoside G-418 (Colberre-Garapin et al., J. Mol. Biol., 150:1, 1981); and hygro, which confers resistance to hygromycin (Santerre et al., Gene, 30:147, 1984).

The nucleic acid molecules of the invention are useful in the diagnosis and treatment of systemic sclerosis and other autoimmune disorders.

### Polypeptides

The invention also includes polypeptides that have a sequence that is encoded by, or is substantially identical to the polypeptides encoded by, the nucleic acids of the invention (e.g., polypeptides that are substantially identical to a polypeptide encoded by any one of SEQ ID NO:1-22). A polypeptide which is “substantially identical” to a given reference polypeptide is a polypeptide having a sequence that has at least 85% identity to the sequence of the given reference polypeptide sequence (e.g., the amino sequence of a polypeptide encoded by any one of SEQ ID NO:1-22). Substantially identical polypeptides can also have a higher percentage identity, e.g., 90%, 95%, 98%, or 99%.

The terms “protein” and “polypeptide” are used herein interchangably to describe any chain of amino acids, regardless of length or post-translational modification (for example, glycosylation or phosphorylation). Thus, the teni “polypeptides of the invention” includes: full-length, naturally occurring proteins of the invention; recombinantly or synthetically produced polypeptides that correspond to full-length naturally occurring proteins of the invention; or particular domains or portions of the naturally occurring proteins. The term also encompasses mature polypeptides that have an added amino-terminal methionine (useful for expression in prokaryotic cells).

The new polypeptides described herein are encoded by any of the nucleic acid molecules described herein and include fragments, mutants, truncated forms, and fusion proteins of polypeptides of the invention. These polypeptides can be prepared for a variety of uses, including, but not limited to, the generation of antibodies, as reagents in diagnostic assays, for the identification of other cellular gene products or compounds that can modulate the activity or expression of nucleic acids or polypeptides of the invention, and as pharmaceutical reagents useful for the treatment of disorders associated with aberrant expression or activity of nucleic acids or polypeptides of the invention.

Polypeptides can be substantially pure polypeptides of the invention, including those that correspond to the polypeptide with an intact signal sequence, and the secreted form of the polypeptide in certain embodiments, the polypeptides are soluble under normal physiological conditions.

The invention also encompasses polypeptides that are functionally equivalent, or substantially equivalent, to polypeptides of the invention. These polypeptides are equivalent to polypeptides of the invention in that they are capable of carrying out one or more of the functions of polypeptides of the invention in a biological system. Polypeptides of the invention can have 60%, 75%, 80%, or even 90% of one or more of the biological activities of the full-length polypeptides of the invention. Such comparisons are generally based on an assay of biological activity in which equal concentrations of the polypeptides are used and compared. The comparison can also be based on the amount of the polypeptide required to reach 50% of the maximal stimulation obtainable.

Functionally equivalent polypeptides can be those, for example, that contain additional or substituted amino acid residues. Amino acid substitution refers to the substitution of one amino acid for another amino acid of the same class. Substitutions may be made on the basis of similarity in polarity, charge, solubility, hydrophobicity, hydrophilicity, and/or the amphipathic nature of the residues involved. For example, a functionally equivalent polypeptide is one in which 10% or fewer of the amino acids in a full-length, naturally occurring polypepticle are replaced by conservative amino acid substitutions, and the functionally equivalent polypeptide maintains at least 50% of the biological activity of the full-length polypeptide. These substitutions can include amino acid residues that represent either a conservative or non-conservative change (or, where more than one residue is varied, possibly both). A “conservative” substitution is one in which one amino acid residue is replaced with another having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The invention includes polypeptides that include one, two, three, five, or more conservative amino acid substitutions, where the resulting mutant polypeptide has at least one biological activity that is the same, or substantially the same, as a biological activity of the wildtype polypeptide.

Polypeptides that are functionally equivalent to polypeptides of the invention can be made using random mutagenesis on the encoding nucleic acids by techniques well known to those skilled in the art. It is more likely, however, that such polypeptides will be generated by site-directed mutagenesis (again using techniques well known to those skilled in the art). These polypeptides may have increased functionality or decreased functionality.

To design functionally equivalent polypeptides, it is useful to distinguish between conserved positions and variable positions. This can be done by aligning the amino acid sequence of a protein of the invention from one species with its homolog from another species. Skilled artisans will recognize that conserved amino acid residues are more likely to be necessary for preservation of function. Thus, it is preferable that conserved residues are not altered.

Mutations within the coding sequence of nucleic acid molecules of the invention can be made to generate variant genes that are better suited for expression in a selected host cell. For example, N-linked glycosylation sites can be altered or eliminated to achieve, for example, expression of a homogeneous product that is more easily recovered and purified from yeast hosts that are known to hyperglycosylate N-linked sites. To this end, a variety of amino acid substitutions at one or both of the first or third amino acid positions of any one or more of the glycosylation recognition sequences which occur, and/or an amino acid deletion at the second position of any one or more of such recognition sequences, will prevent glycosylation at the modified tripeptide sequence (see, for example, Miyajima et al., EMBO J., 5:1193, 1986).

The polypeptides of the invention can be expressed fused to another polypeptide, for example, a marker polypeptide or fusion partner. For example, the polypeptide can be fused to a hexa-histidine tag to facilitate purification of bacterially expressed protein or a hemcagglutinin tag to facilitate purification of protein expressed in eukaryotic cells.

A fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA, 88: 8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺. Nitriloacetic acid-agarose columns and histidine-tagged proteins are selectively eluted with imidazole-containing buffers.

The polypeptides of the invention can be chemically synthesized (for example, see Creighton, “Proteins: Structures and Molecular Principles,” W. H. Freeman & Co., New York, 1983), or, perhaps more advantageously, produced by recombinant DNA technology as described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984).

The invention also features polypeptides that interact with nucleic acids or polypeptides of the invention (and the genes that encode them) and thereby alter the function of nucleic acids or polypeptides of the invention. Interacting polypeptides can be identified using methods known to those skilled in the art. One suitable method is the “two-hybrid system,” which detects protein interactions in vivo (Chien et al., Proc. Natl. Acad. Sci. USA, 88:9578, 199 l). A kit for practicing this method is available from Clontech (Palo Alto, Calif.).

The invention also encompasses small polypeptides that can be used to inhibit specific interactions of two proteins (e.g., a viral membrane protein and a host cell receptor). By virtue of their size, small polypeptides can have many useful properties. For example, they can be membrane-permeable if necessary, or they can be used at high concentration because of their specificity. Small polypeptides can also be used to generate very specific antibodies.

### Detection of Myoplasma hyorhinis

Assays such as immunofluorescence, RIA, ELISA, Western blot, plaque formation, mouse reinfection, gradient isolation, PCR, or Southern blots for DNA are employed to detect M. hyorhinis and infection thereby. The detection process can be carried once, or over multiple rounds to successively improve the efficacy of detecting M. hyorhinis infection.

Growth in cultured cells: M. hyorhinis is can be grown in cultured cells much like a virus. It forms plaques after about 7 days and these plaques can be identified by their ability to stain with M. hyorhinis-specific antibodies. A wide range of cell types (epithelial, fibroblastic) and species (human, monkey, mouse) can be used to culture the organism.

Growth in broth: Most mycoplasmas can be grown in vitro in broth. One can measure turbidity to determine growth characteristics of M. hyorhinis.

Infection of mice: Mice can be infected intranasally with M. hyorhinis and they produce autoantibodies within 6 weeks.

PCR analysis: PCR can be used to detect M. hyorhinis-specific DNA sequences (e.g., SEQ ID NO:1-22) in cells, human tissues, blood, and other samples. This is a very sensitive assay, and can detect M. hyorhinis with a high degree of accuracy and in minute amounts.

Antibodies: M. hyorhinis antibodies can be generated against M. hyorhinis-specific antigens, such as proteins (e.g., vlp gene product). These can be used in radioimmune assays (RIA) or ELISA assays to detect mycoplasma proteins.

Antibodies specific for M. hyorhinis capable of recognizing and binding to Mycoplasma proteins can be used to identify M. hyorhinis in the following assays: Western blotting, immunocytochemistry (immunofluorescence), immunoprecipitation, and immunohistochemistry. Mhyorhinis can be isolated from biological material (e.g., human or animal body fluids), amplified, and cultured in vitro. Such isolates can serve as an M. hyorhinis source for use in any of these above assays. For example, in vitro M. hyorhinis is capable of forming plaques on cultured cell monolayers. Like other Mycoplasma, M. hyorhinis derived from human or mouse is potentially capable of foiling plaques oil agar plates with special media. The mycoplasma that form these plaques can be positively identified as M. hyorhinis using an immunofluorescence assay.

Oligonticleotide DNA probes: Oligonucleotide DNA probes could be made against M. hyorhinis-specific DNA sequences (e.g., SEQ ID NO:1-22) and used to probe DNAs for the presence of this organism.

After treatment with a therapeutic agents of the invention (e.g., antibiotics, antibodies, vaccines, antisense nucleic acids, ribozymes, etc.), assays such as immunofluorescence, RIA, ELISA, Western blot, plaque formation, mouse re-infection, gradient isolation, PCR, or Southern blots for DNA can be employed to determine which substances are useful in treating M. hyorhinis infection. The effectiveness of treatment is correlated with the amount of M. hyorhinis detected using one of the above methods. This process can be carried out once, or over multiple rounds to successively improve the efficacy of the substances for treating M. hyorhinis infection.

### Treating Scleroderma

Mycoplasma hyorhinis infections, for example, those that cause systemic sclerosis, can be treated using a number of approaches. These include antibodies specific for this strain of mycoplasma, which work like a vaccine in that they prevent infection of cells by antibody binding to a surface antigen. Antisense RNAs can be used to disrupt the genetic material of M. hyorhinis. Ribozymes are effective for degrading ribosomes/RNA, and can be used to do so in M. hyorhinis. Antibiotics can be used to treat M. hyorhinis infections. Finally, vaccines can be used to prevent infections or treat infections with M. hyorhinis.

In addition to scleroderma, many diseases with an autoimmune component have mycoplasma, such as Mycoplasma hyorhinis, as an underlying contributor to their pathology. The methods used to treat scleroderma, or to identify compositions that can be used to treat scleroderma, can also be used or adapted to be used to treat other autoimmune diseases.

### Antibodies

Mycoplasma hyorhinis polypeptides (or immunogenic fragments or analogs thereof can be used to raise antibodies useful in the invention; such polypeptides can be produced by recombinant techniques or synthesized (see, for example, “Solid Phase Peptide Synthesis,” supra; Ausubel et al., supra). In general, the peptides can be coupled to a carrier protein, such as KLH, as described in Ausubel et al., supra, mixed with an adjuvant, and injected into a host mammal. Antibodies can be purified by peptide antigen affinity chromatography.

In particular, various host animals can be immunized by injection with a polypeptide of the invention. Host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Useful human adjuvants include BCG (bacille Calmette-Guerini) and Corynebacterium parvum. Polyclonal antibodies are heterogeneous populations of antibody molecules that are contained in the sera of the immunized animals.

Antibodies within the invention include polyclonal antibodies and, in addition, monoclonal antibodies, humanized or chimeric antibodies, single chain antibodies, Fab fragments, F(ab′)₂ fragments, and molecules produced using a Fab expression library.

Monoclonal antibodies, which are homogeneous populations of antibodies to a particular antigen, can be prepared using the polypeptides of the invention described above and standard hybridoma technology (see, for example, Kohler et al., Nature, 256:495, 1975; Kohler et al., Eur. J. Immunol., 6:5 11, 1976; Kohler et al., Eur. J. Immunol., 6:292, 1976; Hammerling et al., “Monoclonal Antibodies and T Cell Hybridomas,” Elsevier, N.Y., 1981; Ausubel et al., supra).

In particular, monoclonal antibodies can be obtained by any technique that provides for the production of antibody molecules by continuous cell lines in culture such as described in Kohler et al., Nature, 256:495, 1975, and U.S. Pat. No. 4,376,110; the human B-cell hybridoma technique (Kosbor et al., Immunology Today, 4:72, 1983; Cole et al., Proc. Natl. Acad. Sci. USA, 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. The hybridoma producing the mAb of this invention may be cultivated in vitro or in vivo. The ability to produce high titers of mAbs in vivo makes this a particularly useful method of production.

Once produced, polyclonal or monoclonal antibodies are tested for specific recognition of polypeptides of the invention by Western blot or immunoprecipitation analysis by standard methods, e.g., as described in Ausubel et al., supra. Antibodies that specifically recognize and bind to polypeptides of the invention are useful in the invention. For example, such antibodies can be used in an immunoassay to monitor the level of a polypeptide of the invention produced by a mammal (for example, to determine the amount or subcellular location of a polypeptide of the invention).

In certain embodiments, antibodies of the invention are produced using fragments or the protein of the invention that lie outside highly conserved regions and appear likely to be antigenic, by criteria such as high frequency of charged residues. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

In some cases it may be desirable to minimize the potential problems of low affinity or specificity of antisera, in such circumstances, two or three fusions can be generated for each protein, and each fusion can be injected into at least two rabbits. Antisera can be raised by injections in a series, preferably including at least three booster injections.

Antisera may also be checked for its ability to immunoprecipitation recombinant proteins of the invention or control proteins, such as glucocorticoid receptor, CAT, or luciferase.

In addition to their therapeutic uses, the new antibodies can be used, for example, in the detection of the polypeptide of the invention in a biological sample as part of a diagnostic assay. Antibodies also can be used in a screening assay to measure the effect of a candidate compound on expression or localization of a polypeptide of the invention. Additionally, such antibodies can be used in conjunction with the gene therapy techniques described to, for example, evaluate normal and/or genetically engineered cells that express nucleic acids or polypeptides of the invention prior to their introduction into the patient. Such antibodies additionally can be used in a method for inhibiting abnormal activity of nucleic acids or polypeptides of the invention.

In other embodiments, techniques developed for the production of “chimeric antibodies” (Morrison et al., Proc. Natl. Acad. Sci. USA, 81:6851, 1984; Neuberger et al., Nature, 312:604, 1984; Talkeda et al., Nature, 314:452, 1984) by splicing the genes from a mouse antibody molecule of appropriate antigen specificity together with genes from a human antibody molecule of appropriate biological activity can be used. A chimeric antibody is a molecule in which t different portions are derived from different animal species, Such as those having a variable region derived from a murine mAb and a human immunoglobulin constant region.

Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al. (J. Acquired Immune Deficiency Syndromes and Human Retrovirology, 14:193, 1997).

Alternatively, techniques described for the production of single chain antibodies (U.S. Pat. Nos. 4,946,778, 4,946,778, and 4,704,692) can be adapted to produce single chain antibodies against polypeptides of the invention. Single chain antibodies are formed by lining the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Antibodies to polypeptides of the invention can, in turn, be used to generate anti-idiotype antibodies that resemble a portion of the protein of the invention using techniques well known to those skilled in the art (see, e.g., Greenspan et al., FASEB J., 7:437, 1993; Nissinoff, J. Immunol., 147:2429, 1991). For example, antibodies that bind to the protein of the invention and competitively inhibit the binding of a binding partner of the protein can be used to generate anti-idiotypes that resemble a binding partner binding domain of the protein and, therefore, bind and neutralize a binding partner of the protein. Such neutralizing anti-idiotypic antibodies or Fab fragments of such anti-idiotypic antibodies can be used in therapeutic regimens.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics, 7:13-21, 1994; see also U.S. Pat. Nos. 5,545,806 and 5,569,825).

The methods described herein in which anti-polypeptide-of-the-invention antibodies are employed may be performed, for example, by utilizing pre-packaged diagnostic kits comprising at least one specific polypeptide-of-the-invention antibody reagent described herein, which may be conveniently used, for example, in clinical settings, to diagnose patients exhibiting symptoms of disorders associated with aberrant expression of nucleic acids or polypeptides of the invention.

An antibody (or fragment thereof) can be conjugated to a therapeutic moiety such as a cytotoxin, a therapeutic agent, or a radioactive agent (e.g., a radioactive metal ion). Cytotoxins and cytotoxic agents include any agent that is detrimental to cells. Examples of such agents include taxol, cytochalasin B, gramicidin D, ethidium bromide, emetine, mitomycini, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof Therapeutic agents include, but are not limited to, antimetabolites (e.g., metlhotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomiiycini C, and cis-dichlorodiamine platinum (II) (DDP) cisplatin), anthracyclines (e.g., daunorubicin {formerly designated daunomycin} and doxorubicin), antibiotics (e.g., dactinomycin {formerly designated actinomycin}, bleomycin, mithramycin, and anthramycin), and anti-mitotic agents (e.g., vincristine and vinblastine).

Conjugated antibodies (i.e., antibodies joined to a moiety of a drug molecule) of the invention can be used for modifying a given biological response. The conjugated drug moiety need not be limited to classical chemical therapeutic agents. For example, the drug moiety can be a protein or polypeptide possessing a desired biological activity. Such proteins include, for example, toxins such as abrin, ricin A, Pseudonomas exotoxin, or Diphtheria toxin; proteins such as tumor necrosis factor, alpha-interferon, beta-interferon, nerve growth factor, platelet derived growth factor, tissue plasminogen activator; and biological response modifiers such as lymphlokines, interleukin-1, interleukin-2, interleukin-6, granulocyte macrophage colony stimulating factor, granulocyte colony stimulating factor, or other growth factors.

Techniques for conjugating a therapeutic moiety to an antibody are well known (see, e.g., Arnon et al., 1985, “Monoclonal Antibodies For Immunotargeting Of Drugs In Cancer Therapy”, in Monoclonal Antibodies And Cancer Therapy, Reisfeld et al., Eds., Alan R. Liss, Inc. pp. 243-256; Hellstrom et al., 1987, “Antibodies For Drug Delivery”, in Controlled Drug Delivery, 2nd ed., Robinson et al., Eds., Marcel Dekker, Inc., pp. 623-653; Thorpe, 1985, “Antibody Carriers Of Cytotoxic Agents In Cancer Therapy: A Review”, in Monoclonal Antibodies '84: Biological And Clinical Applications, Pinchera et al., Eds., pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al., Eds., Academic Press, pp. 303-316, 1985; and Thorpe et al., 1982, immunol. Rev., 62: 119-158). Alternatively, an antibody can be conjugated to a second antibody to form an antibody heteroconjugate as described by Segal in U.S. Pat. No. 4,676,980.

### Aptamers

Aptamers are nucleic acid molecules that bind to specific target molecules based on their three-dimensional conformation rather than hybridization. The aptamers are selected, for example, by synthesizing an initial heterogeneous population of oligonucleotides, and then selecting oligonucleotides within the population that bind tightly to a particular target molecule. Once an aptamer that binds to a particular target molecule has been identified, it can be replicated using a variety of techniques known in biological and other arts, e.g., by cloning and polymerase chain reaction (PCR) amplification followed by transcription. The target molecules can be nucleic acids, proteins, peptides, small organic and inorganic compounds, and even entire micro-organisms.

The synthesis of a heterogeneous population of oligonucleotides and the selection of aptamers within that population can be accomplished using a procedure known as the Systematic Evolution of Ligands by Exponential Enrichment or SELEX. The SELEX method is described in, e.g., Gold et al., U.S. Pat. Nos. 5,270,163 and 5,567,588; Fitzwater et al., (“A SELEX Primer,” Methods in Enzymology, 267:275-301, 1996); and in Ellington and Szostak (“In Vitro Selection of RNA Molecules that Bind Specific Ligands,” Nature, 346:818-22). Briefly, a heterogeneous DNA oligomer population is synthesized to provide candidate oligomers for the in vitro selection of aptamers. This initial DNA oligomer population is a set of random sequences 15 to 100 nucleotides in length flanked by fixed 5′ and 3′ sequences 10 to 50 nucleotides in length. The fixed regions provide sites for PCR primer hybridization and, in one implementation, for initiation of transcription by an RNA polymerase to produce a population of RNA oligomers. The fixed regions also contain restriction sites for cloning selected aptamers. Many examples of fixed regions can be used in aptamer evolution. See, e.g., Conrad et al. (“In Vitro Selection of Nucleic Acid Aptamers That Bind Proteins,” Methods in Enzymology, 267:336-83, 1996); Ciesiolka et al., (“Affinity Selection-Amplification from Randomized Ribooligonucleotide Pools,” Methods in Enzymology, 267:315-35, 1996); Fitzwater, supra.

Aptamers are selected in a 5 to 100 cycle procedure. In each cycle, oligomers are bound to the target molecule, purified by isolating the target to which they are bound, released from the target, and then replicated by 20 to 30 generations of PCR amplification.

Aptamer selection is similar to evolutionary selection of a function in biology. Subjecting the heterogeneous oligonucleotide population to the aptamer selection procedure described above is analogous to subjecting a continuously reproducing biological population to 10 to 20 severe selection events for the function, with each selection separated by 20 to 30 generations of replication.

Heterogeneity is introduced, e.g., only at the beginning of the aptamer selection procedure, and does not occur throughout the replication process. Alternatively, hieterogeneity can be introduced at later stages of the aptamer selection procedure.

Various oligomers can be used for aptamer selection, including, e.g., 2′-fluoro-ribonucleotide oligomers, NH2-substituted and OCH3-substituted ribose aptamers, and deoxyribose aptamers. RNA and DNA populations are equally capable of providing aptamers configured to bind to any type of target molecule. Within either population, the selected aptamers occur at a frequency of 109 to 1013, see Gold et al., (“Diversity of Oligonucleotide Functions,” Annual Review of Biochemistry, 64:763-97, 1995), and most frequently have nanomolar binding affinities to the target, affinities as strong as those of antibodies to cognate antigens. See Griffiths et al., (EMBO J., 13:3245-60, 1994).

Using 2′-fluoro-riboniucleotide oligomers is likely to increase binding affinities ten to one hundred fold over those obtained with unsubstituted ribo- or deoxyribo-oligonucleotides. See Pagratis et al. (“Potent 2′-amino and 2′ fluoro 2′deoxyribonucleotide RNA inhibitors of keratinocyte growth factor” Nature Biotechnology, 15:68-73). Such modified bases provide additional binding interactions and increase the stability of aptamer secondary structures. These modifications also make the aptamers resistant to nucleases, a significant advantage for real world applications of the system. See Lin et al. (“Modified RNA sequence pools for in vitro selection” Nucleic Acids Research, 22:5229-34, 1994); Pagratis, supra.

In the present invention, aptamers can be made that specifically bind to the nucleic acids and polypeptides or the invention, as well as to Mycoplasma hyorhinis itself, using the techniques described herein.

### Antisense Nucleic Acids

Treatment regimes based on an “antisense” approach involve the design of oligonucleotides (either DNA or RNA) that are complementary to mRNA complementary to DNA sequences of the invention (e.g., SEQ ID NO:1-22). These oligonucleotides hind to the complementary mRNA transcripts (target sequences) of the invention and prevent translation. Absolute complementarity, although preferred, is not required. A sequence “complementary” to a portion of an RNA, as referred to herein, means a sequence having sufficient complementarily to be able to hybridize with the RNA in vivo, forming a stable duplex; in the case of double-stranded antisense nucleic acids, a single strand of the duplex DNA may be tested, or triplex formation may be assayed. The ability to hybridize will depend on both the degree of complementarily and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs recently have been shown to be effective at inhibiting translation of mRNAs as well (Wagner, Nature, 372:333, 1984). Thus, oligonucleotides complementary to either the 5′ or 3′ non-translated, non-coding regions of the gene or mRNA could be used in an antisense approach to inhibit translation of endogenous mRNA. Oligonucleotides complementary to the 5′ untranslated region of the mRNA should include the complement of the AUG start codon. Locating target sequences in the untranslated regions of the nucleic acids of the invention can be done by one of skill in the art by following the instructions disclosed herein.

Antisense oligonucleotides complementary to mRNA coding regions are less efficient inhibitors of translation but could be used in accordance with the invention. Whether designed to hybridize to the 5′, 3′, or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides, or at least 50 nucleotides. Target sequences in mRNA coding regions of the nucleic acids of the invention can be located by one of skill in the art by following the instructions disclosed herein.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (as described, e.g., in Letsinger et al., Proc. Natl. Acad. Sci. USA, 86:6553, 1989; Lemaitre et al., Proc. Natl. Acad. Sci. USA, 84:648, 1987; PCT Publication No. WO 88/09810) or the blood-brain barrier (see, for example, PCT Publication No. WO 89/10134), or hybridization-triggered cleavage agents (see, for example, Krol et al., BioTechniques, 6:958, 1988), or intercalating agents (see, for example, Zon, Pharm. Res., 5:539, 1988). To this end, the oligonticleotide can be conjugated to another molecule, for example, a peptide, hybridization triggered cross-linking agent, transport agent, or hybridization-triggered cleavage agent.

The antisense oligonucleotide may include at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhlydroxylmethyl) uracil, 5-carboxymethylaminomethyl-2-thiouridine, 5-carboxymethyl-aminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-theouracil, 2-thiouracil, 4-thiouracil, 5-methyluracil, uracil-5-oxyacetic acid methylester, uracil-5-oxyacetic acid (v), 5-methyl-2-thiouracil, 2-(3-amino-3-N-2-carboxypropl) uracil, (acp3)w, and 2,6-diaminopurine.

The antisense oligonucleotide may also include at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal, or an analog of any of these backbones.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids. Res., 15:6625, 1987). The oligonucleotide is a 2′-0-methylribonucleotide (Inoue et al., Nucl. Acids Res., 15:6131, 1987), or a chimeric RNA-DNA analog (Inoue et al., FEBS Lett., 215:327, 1987).

Antisense oligonucleotides of the invention can be synthesized by standard methods known in the art, e.g., by use of an automated DNA synthesizer (such as are commercially available from Biosearch, Applied Biosystems, etc.). As examples, phosphorothioate oligonucleotides can be synthesized by the method of Stein et al. (Nucl. Acids Res., 16:3209, 1988), and methylphosphonate oligonucleotides can be prepared by use of controlled pore glass polymer supports (Sarin et al., Proc. Natl. Acad. Sci. USA, 85:7448, 1988).

The antisense molecules should be delivered to cells that express nucleic acids or polypeptides of the invention i,n vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense molecule sufficient to suppress translation of endogenous mRNAs. Therefore, a preferred approach uses a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous transcripts of nucleic acids of the invention and thereby prevent translation of the endogenous mRNA. The invention encompasses the construction of an antisense RNA using the complementary strand as a template. For example, a vector can be introduced in vivo such that it is taken up by a cell and directs the transcription of an antisense RNA. Such a vector can remain episomal or become chromosomally integrated, as long as it can be transcribed to produce the desired antisense RNA.

Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include, but are not limited to: the SV40 early promoter region (Bernoist et al., Nature 290:304, 1981); the promoter contained in the 3′ long terminal repeat of Rous sarcoma virus (Yamamoto et al., Cell, 22:787-797, 1988); the herpes thymidine kinase promoter (Wagner et al., Proc. Natl. Acad. Sci. USA, 78:1441, 1981); or the regulatory sequences of the metallothionein gene (Brinster et al., Nature, 296:39, 1988).

Antisense molecules of the invention can be employed to treat or prevent mycoplasma infections. Mycoplasma antigens present on cells or on mycoplasmas that are themselves on cells can be used as indicators of the presence of mycoplasma cells, which can then be specifically targeted for delivery of antisense nucleic acids against mycoplasma. Alternatively, one can target cell-type-specific antigens to deliver effectively antisense nucleic acids to the cells targeted by mycoplasma. One can also treat or prevent mycoplasma infections by using antisense nucleic acids that are highly or uniquely mycoplasma-specific; these antisense nucleic acids can be delivered generally to all cells because they target only mycoplasma.

### Ribozymes

Ribozyme molecules designed to catalytically cleave mRNA transcripts of nucleic acids of the invention (e.g., SEQ ID NO:1-22 depicted in FIGS. 2-10) can be used to prevent translation and expression of mRNA of the invention (see, e.g., PCT Publication WO 90/11364; Saraver et al., Science, 247:1222, 1990). Various ribozymes (e.g., hammerhead ribozymes) that cleave mRNA at site-specific recognition sequences can be used to destroy mRNAs of the invention. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozynes is well known in the art (Haseloff et al., Nature, 334:585, 1988). Preferably, the ribozyme is engineered so that the cleavage recognition site is located near the 5′ end of the mRNA, i.e., to increase efficiency and minimize the intracellular accumulation of non-functional mRNA transcripts. Specifically, the Mycoplasma hyorhinis nucleic acid sequences UUTCTUCUUUGTTGTT (SEQ ID NO:23), TTTTTTUGGTYUGTUTUCU (SEQ ID NO:24), UUUTUUGUUGTTCUTTTTT (SEQ ID NO:25), GCTTTUUUGUCGCGTTTUC (SEQ ID NO:26), UUUUTTUUUGCUUCTTCUGT (SEQ ID NO:27), TTUGTTTUUGUCCTUGTUGU (SEQ ID NO:28), TUUCTTCUGUCGTTGUGCT (SEQ ID NO:29), UUGUCCTUGUTGTCCUTCUG (SEQ ID NO:30), and GTUUUTUCUUGUUTTTTTTCTT (SEQ ID NO:31) are good targets for ribozymes.

The ribozymes of the present invention also include RNA endoribonucleases (hereinafter “Cech-type ribozymes”), such as the one that occurs naturally in Tetrahymena thermophilia (known as the IVS or L-19 IVS RNA), and which has been extensively described by Cech and his collaborators (Zaug et al., Science, 224:574, 1984; Zaug et al., Science, 231:470, 1986; Zug et al., Nature, 324:429, 1986; PCT Application No. WO 88/04300; and Been et al., Cell, 47:207, 1986). The Cech-type ribozymes have an eight base-pair sequence that hybridizes to a target RNA sequence, whereafter cleavage of the target RNA takes place.

As in the antisense approach, the ribozymes can be composed of modified oligonucleotides (e.g., for improved stability, targeting, etc.), and should be delivered to cells which express nucleic acids or polypeptides of the invention in vivo. A preferred method of delivery involves using a DNA construct “encoding” the ribozyne under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

### RNA Interference

A stretch of sequence that is not likely to form stable secondary structures can be chosen for the generation of short interfering RNAs (siRNAs) for the purpose of inhibiting or decreasing expression of mRNA into more than one protein. Another guideline is that the GC content of the siRNA oligo be in the range of 30% and 70%. In one aspect, RNAi can be used to target specific sequences within the nucleic acids of the invention. RNAi can be used to target, for example, SEQ ID NOs:1-22. The complement or RNA equivalent of a nucleic acid of the invention can also act as RNAi agents (e.g., siRNA duplexes). The inclusion of a nucleic acid of the invention, their complement, and/or their RNA equivalent in larger molecules such as plasmid-based systems can also be used to generate an RNAi response. As described below, siRNAs can suppress or decrease the expression of the targeted open reading frames encoding a polypeptide of the invention.

RNAi is a remarkably efficient process whereby double-stranded RNA (dsRNA) induces the sequence-specific degradation of homologous mRNA in animals and plant cells (Hutvagner and Zamore, Curr. Opin. Genet. Dev. 12:225-232, 2002; Sharp, Genes Dev. 15:485-490, 2001). In mammalian cells, RNAi can be triggered by 21-nucleotide (nt) duplexes of small interfering RNA (siRNA) (Chiu et al., Mol. Cell. 10:549-561, 2002; Elbashir et al., Nature 411:494-498, 2001), or by micro-RNAs (miRNA), functional small-hairpin RNA (shRNA), or other dsRNAs which are expressed in vivo using DNA templates with RNA polymerase III promoters (Zeng et al., Mol. Cell 9:1327-1333, 2002; Paddison et al., Genes Dev. 16:948-958, 2002; Lee et al., Nature Biotechnol. 20:500-505, 2002; Paul et al., Nature Biotechnol. 20:505-508, 2002; Tuschl, T., Nature Biotechnol. 20:440-448, 2002; Yu et al., Proc. Natl. Acad. Sci. USA 99(9):6047-6052, 2002; McManus et al., RNA 8:842-850, 2002; Sui et al., Proc. Natl. Acad. Sci. USA 99(6):5515-5520, 2002).

Suppliers of RNA synthesis reagents and synthesized RNA oligos are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

The nucleic acid molecules or constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region in the mRNA of a DNA of the invention, and the other strand is identical or substantially identical to the first strand. The dsRNA molecules of the invention can be chemically synthesized, or may transcribed in vitro from a DNA template, or in vivo from, e.g., shRNA.

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

Crosslinking can be employed to alter the pharmacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al. (Drug Deliv. Rev. 47(1), 99-112, 2001 (describes nucleic acids loaded to polyalkylcyanoacrylate (PACA) nanoparticles); Fattal et al., J. Control Releas, 53(1-3)137-43, 2001 (describes nucleic acids bound to nanoparticles); Schwab et al., Ann. Oncol. 5 Suppl. 4:55-8, 1994 (describes nucleic acids linked to intercalating agents, hydrophobic groups, polycations or PACA nanoparticles); and Godard et al., Eur. J. Biochem. 232(2):404-10, 1995 (describes nucleic acids linked to nanoparticles).

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using 3H, 32P, or other appropriate isotope.

### Nucleic Acid Vaccines

A “nucleic acid vaccine” is a vaccine whose active ingredient is at least one isolated nucleic acid that encodes a polypeptide antigen. Nucleic acid vaccination involves the direct introduction by needle injection, particle bombardment, or other such known methods, into host organism tissues of a plasmid DNA (or an RNA) capable of causing (in antigenic protein to be expressed directly inside the transfected cells. Like viral infection the host's own intracellular processes are used to synthesize proteins, sometimes even resulting in secondary modifications of the synthesized antigen. The plasmid vectors introduced into host cells possess the elements required for expression in eukaryotic organisms. Thus, the plasmids used in nucleic acid vaccination are capable of in situ expression of antigenic proteins. Nucleic acid vaccines can be used both prophylactically in naïve individuals, or therapeutically in individuals already infected.

The nucleic acids employed in the nucleic acid vaccines of the invention include at least three components: (1) a mycoplasma coding sequence, (2) a mammalian transcriptional promoter operably linked to the coding sequence for expression of the mycoplasma coding sequence, and (3) a mammalian polyadenylation signal operably linked to the coding sequence to terminate transcription driven by the promoter. In the context of this invention a “mammalian” promotor or polyadenylation signal is not necessarily a nucleic acid sequence derived from a mammal. For example, some mammalian promoters and polyadenylation signals are viral in origin. In addition to the three components listed above, nucleic acids used in nucleic acid vaccines may additionally include some of the following: enhancer elements, splicing signals, termination and polyadenylation signals, viral replicons, and bacterial plasmid sequences.

For example, nucleic acid encoding a mycoplasma antigen of interest can be inserted into expression vectors that are commercially available (e.g., Invitrogen Catalogs, 1998). Yasutomi et al. (J. Virol., 70:678-681, 1996) have also described vectors specifically constructed for nucleic acid vaccines.

Nucleic acid vaccines provide several advantages over conventional vaccines due to the stability of DNA under a variety of conditions. This stability allows nucleic acid vaccines to be stored and shipped easily. Also, because they do not require adjuvants, they may be more efficient and less expensive to produce. Nucleic acid vaccines are safer than vaccines based on live vectors (e.g., viruses, bacteria). Furthermore, they lead to the production of more native antigen conformations, they allow easier modification of the amino acid sequence of antigen of interest, and they allow the codelivery and expression of other antigens or polypeptide adjuvants.

The invention encompasses nucleic acid vaccines made using DNA sequences specific to M. hyorhinis sequences and introduced into the skin of individuals. The antibodies formed are able to target any mycoplasma antigens. Most effective are surface molecules, such as variable lipoprotein (vlp) (e.g., human, mouse), although antibodies can be made against any and all pathogen proteins. This method of treating mycoplasma infection is effective because DNA is delivered to the host cell, and then the host cell uses its pre-existing ability to present antigens and make antibodies to them. This method has been successfully employed for numerous viruses.

Nucleic acid vaccines of the invention can be administered by a variety of means. Injection or inoculation can be employed. Or, nucleic acids can be encapsulated in microparticles (e.g., U.S. Pat. No. 5,620,896) that are subsequently delivered by conventional particle bombardment (e.g., Accell II™ from PowderJect Vaccines, inc., Middleton, Wis.; Helios Gene Gun from BioRad). Additional information on particle bombardment can be found in sources including the following: Yang et al., P.N.A.S USA, 87:9568, 1990; Yang, CRC Crit. Rev. Biotechnol., 12:335, 1992; Richmond et al., Virology, 230:265-274, 1997; Mustafa et al., Virology, 229:269-274, 1997; Livingston et al., Infect. Immun., 66:322-329, 1998; and Cheng et al., P.N.A.S. USA, 90:4455, 1993.

Alternatively, a nucleic acid vaccine of the invention can be delivered by inoculation. Mucosal inoculation can be accomplished by a variety of methods, including nucleic acid-containing nose-drops, inhalants, suppositories, microspheres, or encapsulated in poly(lactide-co-glycolide) (PLG) micropaiticles by a solvent extract technique (e.g., Jones et al., Infect. Immun., 64:489, 1996; Jones et al., Vaccine, 15:814, 1997).

Regardless of the route of administration, an adjuvant can be administered before, during, or after administration of the nucleic acid to improve the efficiency of delivery and effectiveness of prophylactic or therapeutic functioning of the nucleic acid vaccine.

### Antibiotics

Antibiotics can be used to treat Mycoplasma pneumonia infections. Patients can be treated with antibiotics used to treat bacteria (e.g., tetracycline) but the treatment is much longer (for several months) and the concentration of the drugs usually higher (2-fold or greater). Scleroderma can be treated by the application of antibiotics to the organism (e.g., human, mouse, domesticated mammals, or birds) suffering from Mycoplasma hyorhinis infection. Antibiotics and their physiologically acceptable salts and solvates can be formulated for administration by inhalation or insufflation (either through the mouth or the nose), intravenous oral, buccal, parenteral, rectal administration.

Antibiotics that can be used to treat M. hyorhinis include quinolones (e.g., ciprofloxacin, levofloxacin), tetracycline and its derivatives doxycycline and minocycline, erythromycin and its derivatives clarithromycin and azythromycin, third-generation cephalosporins (e.g., ceftriaxone, cefotaxim), clindamycin, vancomycin, tiamulin, and lincocin. Other antibiotics can also be employed effectively.

Antibiotic treatment of systemic sclerosis can be administered for various periods of time, depending on such factors as dosage and patient response to treatment. For example, antibiotic treatment can be applied for several months, with patients being reassessed periodically and doses adjusted according to patients' conditions. Dosages can be lowered for patients sensitive to an antibiotic or increased for patients resistant to antibiotics. The antibiotic regimen can be discontinued after the infection is no longer detected in the infected patient. Antibiotics with potentially higher toxicity, such as minocycline, can be started at lower doses (e.g., 50 mg twice daily), and the dose can be increased (e.g., to 100 mg twice daily). Conversely, antibiotics with potentially lower toxicity can be started at higher doses. Dosage can also depend on what organs are involved. For example, acute lung or intestinal involvement may require antibiotic treatment to start at a higher dose (e.g., ciprofloxacin at 500 mg twice daily), and this dose may be tapered down with time.

For patients with long-term infections intravenous antibiotic treatments (e.g., clindamycin, lincocin) administered daily for roughly a week can be effective. For example, a regimen of clindamycin can consist of the following: on days 1 and 2, 300 mg in 250 cc 0.9 saline dripped over roughly an hour; on days 3 and 4, 600 mg; and on days 5 and subsequent, 900 mg; a dose of 1200 mg of clindamycin can be administered orally once a week; an intramuscular injection of 300-600 mg can be administered once a week. Such a regimen can be repeated in a variety of patterns of administration optimized to each patient's medical requirements (e.g., seven day treatment repeated every five weeks for a total of four times), and the patient can be reassessed after each administration to optimize treatment further. Treatment with antibiotics can also be maintained for multiple months, or even indefinitely, in the case of persistent infection, although it is important to reassess the patient's condition periodically. Intravenous antibiotic therapy can be followed by oral or intramuscular injection therapy, or vice versa. For example, an initial intravenous regimen. Such as that described above, can be followed by oral therapy with minocycline (e g., 100 mg once or twice daily) or tetracycline (e.g., 250-500 mg twice daily on alternate days).

Based on the results described herein with scleroderma, antibiotics can be used similarly to treat or prevent other autoimmune diseases.

### Screening Assays

Compounds that specifically bind to nucleic acids or polypeptides of the invention, and thus can be used to treat systemic sclerosis, can be identified using any standard binding assay. For example, candidate compounds can be bound to a solid support. A nucleic acid or polypeptide of the invention is then exposed to the immobilized compound and binding is measured (European Patent Application 84/03564).

In one embodiment, the invention provides assays for screening candidate or test compounds that bind with or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam, Anticancer Drug Des., 12:145, 1997).

Examples of methods useful for the synthesis of molecular libraries can be found in the art, for example in: DeWitt et al., Proc. Natl. Acad. Sci. USA, 90:6909, 1993; Erb et al., Proc. Natl. Acad. Sci. USA, 91:11422, 1994; Zuckerinai et al., J. Med. Chem., 37:2678, 1994; Cho et al., Science, 261:1303, 1993; Carrell et (al., Angew. Chem. Int. Ed. Engl., 33:2059, 1994; Carell et al., Angew. Chem. Int. Ed. Engl., 33:2061, 1994; and Gallop et al., J. Med. Client., 37:1233. 1994.

Libraries of compounds can be presented in solution (e.g., Houghten, Bio/Techniques, 13:412-421, 1992), or on beads (Lam, Nature, 354:82-84, 1991), chips (Fodor, Nature, 364:555-556, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA, 89: 1865-1869, 1992) or phage (Scott and Smith, Science, 249:386-390, 1990; Devlin, Science, 249:404-406, 1990; Cwirla et al., Proc. Natl. Acad. Sci. USA, 87:6378-6382, 1990; and Felici, J. Mol. Biol., 222:301-310, 1991).

In one embodiment, an assay is a cell-based assay in which a cell that expresses a membrane-bound form of a polypeptide of the invention, or a biologically active portion thereof, on the cell surface is contacted with a test compound and the ability of the test compound to bind with the polypeptide is determined. The cell, for example, can be a yeast cell or a cell of mammalian origin. Determining the ability of the test compound to bind with the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of a polypeptide of the invention, or a biologically active portion thereof, on the cell surface with a known compound that binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind with the polypeptide or a biologically active portion thereof as compared to the known compound.

In another embodiment, the assay involves assessment of an activity characteristic of the polypeptide, wherein binding of the test compound with the polypeptide or a biologically active portion thereof alters (i.e., increases or decreases) the activity of the polypeptide.

### Mycoplasma and Other Autoimmune Diseases

No autoimmune disease is characterized by a single autoantibody. Essentially all of these diseases share reactivity to a common subset of autoantigens. Each disease is usually characterized by a panel of autoantigens that are reactive in the sera of the afflicted individuals. For example, >40% of scleroderma patients react with centrosomes while only 10% of those with rheumatoid arthritis and diabetes react with centrosomes. This type of information acquired from studies with multiple autoantigens provides a framework for characterizing the diseases. For this reason, it is thought that artoreactivity in all autoimmune diseases come from a common insult. It is possible that mycoplasma hyorhinis is an inducer of antibodies in all autoimmune diseases.

### Effective Dose

Toxicity and therapeutic efficacy of the molecules disclosed in the invention (e.g, nucleic acids, polypeptides, ribozymes, vaccines, aptamers, or antibodies) and the compounds that modulate their expression or activity can be determined by standard pharmaceutical procedures, using either cells in culture or experimental animals to determine the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Polypeptides or other compounds that exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethanie, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

The compounds may also be formulated in rectal compositions Such as suppositories or retention enemas, for example, containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

The therapeutic compositions of the invention can also contain a carrier or excipient, many of which are known to skilled artisans. Excipients that can be used include buffers (for example, citrate buffer, phosphate buffer, acetate buffer, and bicarbonate buffer), amino acids, ulea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration. For example, administration can be parenteral, intravenous, subcutaneous, intramuscular, intracranial, intraorbital, opthalmic, intraventricular, intracapsular, intraspinal, intracistemal, intraperitoneal, transmucosal, or oral. The modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.” It is expected that the preferred route of administration will be intravenous.

It is recognized that the pharmaceutical compositions and methods described herein can be used independently or in combination with one another. That is, subjects can be administered one or more of the pharmaceutical compositions, e.g., pharmaceutical compositions comprising a nucleic acid molecule or protein of the invention or a modulator thereof, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both a cytotoxic agent which selectively kills aberrant cells and an antibody (e.g., an antibody of the invention) which can, in one embodiment, be conjugated or linked with a therapeutic agent, a cytotoxic agent, an imaging agent, or the like.

The invention will be further described in the following examples, which do not limit the scope of the invention described in the claims.

### EXAMPLES

The invention is further described in the following examples, which do not limit the scope of the invention described in the claims. The general experimental procedures, used to investigate Mycoplasma hyorhinis, are described first. Specific examples follow.

### Antibodies

Mouse monoclonal antibodies to Mycoplasma hyorhinis variable lipoprotein were a gift of Dr. K. S. Wise of the University of Missouri. Rabbit polyclonal antibodies to Mycoplasma hyorhinis were purchased from Cortex Biochem (San Leandro, Calif.). A rabbit anti-pericentrin antibody was used as a centrosome marker (Doxsey et al., Cell 76:639-650, 1994). A rabbit anti-calnexin antibody (Stressgen, Victoria, BC Canada) and a mouse monoclonal IgG2a anti-BiP antibody (BD Biosciences, Franklclini Lakes, N.J.) were used as markers for the endoplasmic reticulum.

### Mycoplasma Stocks

Two methods were used to prepare stocks of murine mycoplasma. Supernatants of Vero cell monolayers with plaques were harvested 7 days after inoculation with 103 pfu of mycoplasma/T150 flask. After a preliminary centrifugation in closed 50 ml tubes at 1075×g (2000 rpm), 4° C. for 20 min, infected supernatants are loaded gently on 1.5 ml cushions of 70% renografin in PBS, added at the bottom of centrifuge tubes (25×89 mm, Beckman Instruments, Inc., Palo Alto, Calif.), and centrifuged at 28,000 rpm in an SW28 rotor (Beckman Instruments, Inc., Palo Alto, Calif.) at 4° C. for 90 min. The fraction harvested at the renografin-media interface is subsequently added to the top of a 20-70% continuous renografin gradient and centrifuged at 28,000 rpm in an SW28 rotor at 4° C. for 12 h. The refractive index of gradient fractions is determined using a refractometer. Mycoplasma stocks in sucrose, renografin or DMEM are maintained at −80° C. A second method used to further purify mycoplasma from renografin fractions. Mycoplasma was grown in SP3 broth and plated on SP3 bacterial plates. Colonies containing mycoplasma were isolated, regrown, and re-isolated a total of three times before introduction into mice.

### Cell Lines

NIH3T3 is a murine fibroblast cell line; SP2 is a murine plasmacytoma; Vero is a fibroblast cell line derived from African green monkey kidney; 293T is a human epithelial kidney cell line. Cells were propagated in DMEM (Gibco, Grand Island, N.Y.) supplemented with 100 U/ml of penicillin G (Gibco, Grand Island, N.Y.), 100 mg/ml streptomycin sulfate (Gibco, Grand Island, N.Y.), 2 mM L-glutamine (Gibco, Grand Island, N.Y.), and 10% heat inactivated (56° C., 30 min), certified mycoplasma-free fetal bovine serum (Gibco, Grand Island, N.Y.). All tissue culture reagents, trypsin (Gibco, Grand Island, N.Y.) included, were tested for mycoplasma contamination using a commercially available PCR kit (Stratagene, La Jolla, Calif.).

### Plaque Assay

To obtain organ homogenates, mouse spleens were placed in 1 ml of cold media and homogenized in a mortar and pestle device. The spleen homogenates were centrifuged at 1075×g (2000 rpm) at 4° C. for 20 min to obtain clarified supernatants. Freshly confluent cell monolayers were grown on 6-well plates. Ten-fold serial dilutions of infectious agent inoculi were prepared. Diluted inoculi contained in 0.1 ml were added to 1 ml fresh media per well and cells were incubated for 90 min at 37° C. Monolayers were overlayed with 4 ml/well EMEM media (BioWhittacker, Walkersville, Md.) containing 0.5% agarose (SeaKem, BMA Rockland, Me.) and supplemented with 5% certified mycoplasma-free fetal calf serum, 100 U/ml of penicillin G, 100 mg/ml streptomycin sulfate, 2 mM L-glutamine and 2.5 U/ml nystatin (Gibco, Grand Island, N.Y.). Monolayers were incubated at 37° C. and six days later 2 ml of 0.5% agarose in EMEM containing 0.3 mg Neutral Red (Sigma, St. Louis, Mo.) were added to each well. Plaques were counted one day later. Ciprofloxacin (ICN Biomedicals, Irvine, Calif.) was used at a concentration of 0.5 mg/ml. Doxicycline (Sigma, St. Louis, Mo.) was used at a concentration of 1 mg/ml. Antibiotic was added to mycoplasma infected cultures for 24 hours or 7 days and supernatants were subsequently titrated by plaque assay.

### Cloning, PCR, Sequencing, and Sequence Alignment

DNA was extracted from gradient purified murine mycoplasma, from lyophilized Mycoplasma hyorhinis (supplied by ATCC), and from colony purified material using a proteinase JK digestion protocol (Qiagen Inc., Valencia, Calif.). Genonic mycoplasma DNA was cleaved with EcoRI or HindIII (N.E.B., Beverly, Mass.), and the digests were gel-purified and randomly cloned into pUC18 vector. Alternately, gel purified PCR amplification products could be cloned using a TA cloning system (Invitrogell, San Diego, Calif.). Plasmid DNA from individual clones was purified using QIAprep columns (Qiagen Inc., Valencia, Calif.), and the nucleotide sequences were determined using adequate vector primers at the DNA Sequencing Facility of the University of Massachusetts Medical Center. NCBI databases were searched for homologous sequences using BLASTX (http://www.ncbi.nlm.nih.gov/blast). Comparing each mycoplasma sequence to public databases resulted in a list of alignments. Primers were designed based on these alignments and the corresponding genes were specifically amplified from mouse and pig Mycoplasma hyorhinis strains and compared. Primer sequences are as follows: for the 16s gene 5′-GGTTAAGTCCTGCAACGAGC-3′ and 5′-GTTAACTCACCGACTTTGGG-3′; for the tuf gene 5′-GGCTTGGTGCTGCTCAAATGGA-3′ and 5′-CCTACAGTTCTACCACCTTCACGG-3′; for the putative methylase gene 5′-GATAATACAAGAAGTGGTTTATTGC-3′ and 5′-AAAACTTTCCAACTCGAGTTATATCC-3′; for the putative dehydrogenase gene 5′-TGAAGAAACTTTAGATGTTTCAACAACTCC-3′ and 5′-TCCTGTTGATTTTTCTACATTC-3′; for the putative permease gene 5′-CCAGTTTTTGTAGATATTAAAGAAATCG-3′ and 5′-CTGTAGCTGCAAAAAATCC-3′; for the tpi gene 5′-ATTTGGATTTTGCAATTGC-3′ and 5′-TTTTCTTGCGAAACTGAGCCAACC-3′.

Each PCR reaction was prepared in a laminar flow PCR hood (AirClean, Raleigh, N.C.) and used 50 pmol of each primer, 5 U of HotStarTaq polymerase (Qiagen Inc., Valencia, Calif.), 100 mM of each deoxynucleotide triphosphate, in 67 mM Tris buffer (pH8.8), 4 mM MgCl₂, 16 mM (NH₄)2SO₄, 10 mM 2-mercaptoethanol, and 100 g/ml bovine selumalbumin (BSA).

PCR amplifications were performed at 95° C. for 5 min (1 cycle), 94° C. for 1 min, 50° C. for 2 min, 72° C. for 1 min (40 cycles) and 72° C. for 5 min (1 cycle). Amplifications were carried out in an MJ Theriocycler (MJ Research, Watertown, Mass.). Gel purified PCR amplification products were cloned using a TA cloning system (Invitrogel, Carlsbad, Calif.) according to the manufacturer's instructions. PCR reactions were performed by two independent investigators.

For example, a 280 bp sequence of the tuf gene was amplified as follows: 95° C. for 5 min (1 cycle), 94° C. for 1 min, 45° C. for 2 min, 72° C. for 1 min (40 cycles), and 72° C. for 5 min (1 cycle). A 240 bp sequence of the 16S rRNA gene was amplified as follows: 95° C. for 5 min (1 cycle), 94° C. for 1 min, 60° C. for 2 min, 72° C. for 1 min (40 cycles), and 72° C. for 5 min (1 cycle). PCR reaction mix preparation, DNA sample preparation and addition, amplification, and plasmid preparation were carried out in four different laboratories to prevent cross-contamination. To control for DNA quality in human biopsy material, a 350 bp sequence of the human alpha-tubulin gene was amplified. Amplifications were carried out in an MJ Thermocycler.

### Immunoblotting

Fifty micrograms of recombinant fusion protein was heated to 90° C. in 200 ml of sample buffer for 3 min, subjected to electrophoresis on a 12% sodium dodecyl sulfate (SDS)-polyacrylamide curtain gel (without wells), and transferred to Immobilon membrane (Millipore Corporation, Bedford, Mass.) at 150 mA for 1 hour in a semidry blotter (Fisher Biotech, Pittsburgh, Pa.) at room temperature (RT). The membrane was incubated in Tris buffered saline (TBS) pH 7.4 containing 1% bovine serum albumin (BSA, Sigma, St. Louis, Mo.) at RT for 60 minutes or at 4° C. overnight. The membrane was then washed in TBS with 0.05% Tween (TBST) for 20 minutes and subsequently incubated with dilution series of mouse sera starting at dilutions of 1/40 in TBST-BSA at RT for 60 minutes. A multiwell miniblotter (Integrated Separation Systems, Mass.) was used to probe 56 sera at one time. The incubation step was followed by washing in TBST for 60 minutes with three changes. The membrane was incubated for 60 minutes at room temperature with horse radish peroxidase (HRP)-coupled anti-mouse secondary antibodies (Amersham, Piscataway, N.J.) and washed in TBS. Bound secondary antibodies were detected by chemiluminescence according to the manufacturers protocol (Kirkegaard & Perry Laboratories, Gaithersburg, Md.).

### Immunofluorescence

Immunofluorescence assays were preformed as described (Gavanesen et al., J. Clin Immunol 19:166-171, 1999). Briefly, cultured mouse NIH/3T3 cells or Vero cells were grown on coverslips, washed in phosphate-buffered saline (PBS), pH 7.35 and fixed in methanol at −20° C. for 10 minutes or in 4% paraformaldehide (Electron Microscopy Science, Ft. Washington, Pa.) in PBS at room temperature (RT) for 30 minutes. Fixed cells were washed in PBS containing 0.1% BSA and 0.25% Triton X-100 (PBSAT) and subsequently incubated with freshly prepared serially diluted mouse sera in a humidified chamber for 40 min at RT. Marker antibodies were incubated for 20 minutes at RT. Cells were washed 5 times with PBSAT and incubated for 20 minutes with FITC-coupled goat anti-rabbit and anti-mouse secondary antibodies. Cy3-coupled donkey anti-mouse antibodies specific for IgG (H+L) or IgM were purchased from Jackson ImmunoResearch Laboratories (West Grove, Pa.), as were Cy3- and biotin-coupled goat anti-mouse antibodies specific for Fcg used to detect autoantibodies of IgG isotype, or Cy3-coupled anti-goat antibody used for signal amplification in some experiments. Rat anti-mouse IgG1, IgG2a, and IgG2b purchased from Pharmingen (San Diego, Calif.), rabbit and rat anti-mouse IgG1 and IgG2b purchased from Zymed (San Francisco, Calif.) were used to define autoantibody subclasses. Secondary antibodies were washed 5 times with PBSAT. Nuclear DNA was labeled with 5 pg/ml dapi in PBS for 1 minute. Cells were washed once with PBS, briefly with water and mounted using (Molecular Probes, Eugene, Oreg.). Slides were examined on an Axiophot microscope (Zeiss, Germany). Comparisons between infected and uninfected mouse groups were scored open label. Results were confirmed blindly by one or two investigators.

### Electron Microscopy

Like immunofluorescence, electron microscopy (EM) can be used to identify Mycoplasma hyorhinis in the skin and other organs of the body. EM can also be used for the same purposes as above. In addition, the resolution of the EM is much higher and it is possible that this technique can provide much information about the characteristics of this form of mycoplasma and the cells that it invades.

After plaque-formation, monolayers were fixed and embedded in situ. Mycoplasma was pelleted from enriched gradient fractions, fixed and embedded. Fixation was performed in 4% paraformaldehide (Electron Microscopy Science, Ft. Washington, Pa.) in PBS at RT for 30 minutes. Samples were processed for electron microscopy as described (Doxsey et al., Cell, 76:639-650, 1994). Thin sections (500-700 nm) of the embedded samples were cut on a Sorvall ultramicrotome, stained with uranyl acetate and lead citrate (Ted Pella Incorporated, Redding, Calif.) and viewed in a JOEL electron microscope (Doxsey et al., Cell, 76:639-650, 1994).

### Statistical Analysis

Statistical analysis was performed using an Epi Info 6.1 software package. Comparisons between autoantibody titers in infected and uninfected mice were performed using the Mann-Whitney test. The Student t test, two-tailed and unequal variances was used where indicated.

### Histology

Immunoperoxidase labeling and immunofluorescenice (IF) labeling using Mycoplasma hyorhinis-specific antibodies is used to detect Mycoplasma hyorhinis in the skin (and other infected organs) of scleroderma patients. Such antibody assays can provide a prognostic indicator of disease onset or progression allowing earlier and perhaps more efficacious treatments. This approach can also reveal information on the cell types that are infected with M. hyorhinis. In addition, this information can be used to facilitate targeting of immune components (cells, complement, etc.) that are involved in the autoimmune response.

### Example 1

### Discovery of Mycoplasma hyorhinis in Mouse Model of Scleroderma

To understand the mechanism of autoantibody production in scleroderma, we examined a putative mouse model for scleroderma that exhibited centrosome autoreactivity. To our surprise, centrosome autoreactivity did not segregate with disease features but was randomly distributed in the population.

To test the possibility that an infectious agent accounted for autoantibody production, we housed centrosome-negative mice with infected mice. Centrosome antibodies were detected in these mice within 2 weeks while control mice housed in the same room remained negative (data not shown). Specifically, sera react with the recombinant centrosome autoantigen pericentrin by Western blotting, and recognize centrosomes by immunofluorescense.

Cells were prepared from spleens of mice and placed over cultures of COS (monkey) cells embedded in agar. Virus-like plaques formed in plates with infected but not uninfected spleens. Supernatants from these plates were able to generate plaques when replated. The agent responsible for plaque formation was plaque-purified, collected from cell supernatants and banded on density gradients. Antibodies raised against the banded material stained the periphery of the plaques, where infectious agents are known to localize. DNA was purified from the banded material and nucleotide sequences revealed homology to several genes of a single organism (6 different genes), Mycoplasma hyorhinis. The homologous genes are 16S ribosomal subunit, Vlp (variable lipoprotein), pyruvate dehydrogenase, methylase, Tuf (transcription elongation factor), and triosoplosphate isomerase (tpi).

There are more than 180 known strains of mycoplasma. They have long been considered contaminants of cell cultures. The murine M. hyorhinis of the invention is novel. Previously, only porcine M. hyorhinis had been identified. Furthermore, only 2 known strains of mycoplasma were known to be intracellular, making the intracellular location of M. hyorhinis highly unusual. The known intracellular forms are poorly understood, but they are known to induce polyclonal B-Cell activation, which is consistent with autoantibody production.

Other features confirmed the presence of mycoplasma. Electron microscopy on fractions obtained from banded material revealed bacteria-like microorganisms without cell walls typical of mycoplasma and plaques in cell monolayers stained positively with mycoplasma antibodies.

To determine whether M. hyorhinis was the agent responsible for generating centrosome autoantibodies, we introduced into the nasal passages of mice fractions containing the banded material. The presence of autoantibodies in the sera of mice was monitored by Western blotting for the centrosome protein pericentrin (Doxsey et al., Cell, 76:639-650, 1994) and by immunofuorescence staining of centrosomes. Mice that received fractions prepared from cells infected with the agent developed centrosome reactivity (n=37) while fractions from uninfected cells did not (n=25). Similar results were obtained in both male and female mice, in several mouse strains (Balb.c/SvJ, 129/J, and C57b1/6) tested and when fractions prepared in different ways. In cell culture medium, the half life of M. hyorhinis was relatively short, about 6 hours. Moreover, M. hyorhinis obtained commercially (ATTC) produced centrosome autoantibodies, albeit with lower efficiency. This is likely due to the method of preparation (in vitro culturing of the organism outside cells) and the method of storage (lyophilized powder) of the commercially available material.

The ability of M. hyorhinis to generate centrosome autoantibodies in mice indicated that a similar mechanism could be operating in human scleroderma. As an initial test of this idea, we prepared primers to regions of M. hyorhinis genes that were most homologous between pig and mouse and attempted to amplify homologous sequences from skin samples obtained from individuals with scleroderma using PCR. The 16S ribosomal submit, “tuf” elongation factor, and tpi were used. PCR products were amplified from nearly every scleroderma sample examined with both sets of primers. These included samples from three different sources analyzed in three different experiments by two separate research groups. All PCR products that were cloned and sequenced (n=38) were most homologous to murine and porcine M. hyorhinis sequences. In contrast, PCR products were never detected in samples of skin lesions characterized by inflammation and lymphocytic infiltration, but lacking autoimmune features or in samples from normal human skin. In all tissue samples, tubulin was reproducibly amplified demonstrating that similar amounts of DNA were present in all and all supported PCR-based DNA.

The invention is based, in part, on the experimental induction of centrosome autoantibodies by M. hyorhinis infection. Consequently, the invention includes the development of a new mouse model for studying the mechanisms of autoantibody production, the identification of a novel human mycoplasma, the discovery of that mycoplasma in scleroderma skin lesions, and the discovery that this novel mycoplasma is involved in triggering autoantibody production that causes pathogenesis in scleroderma.

### Example 2

### Mouse Rearing, Infection, and Antibiotic Treatment

Balb.c/SvJ, C57BL/6J and 129/J mice were purchased from the Jackson Laboratories (Bar Harbor, Me.), Charles River Laboratories (Wilmington, Mass.) and Taconic Farms (Germantown, N.Y.). Uninfected mice were housed in a pathogen free facility, while infected mice were housed under biocontainment conditions at the University of Massachusetts Medical Center. Sera were also obtained from mouse colonies housed under conventional conditions at the University of Massachusetts Medical Center and the University of Connecticut Health Center. Six week old male or female mice were pre-bled and anesthetized each with 1 mg Nembutal (Abbott Laboratories, Chicago, Ill.). Mice were infected intranasally with 10³-10⁹ pfu/ml of a 1× or 4× plaque-purified inoculum of infectious agent that had been partially purified on a renografin gradient (Squibb Diagnostics, New Brunswick, N.J.), sedimented on a cushion of 70% sucrose in PBS or grown in broth culture following colony purification. For some experiments, infectious particles were delivered orally, intravenously, and intraperitoneally using conventional procedures.

Centrosome autoantibody production was observed at mycoplasma titers between 10⁴-10⁹ plaque-forming units (pfu). The efficiency of autoantibody production in experimental groups (50-75% of the animals) did not increase at higher titers and was not observed below 104 pfu. Similar results were observed in many mouse strains including Balb.c/SvJ, C57BL/6J and 129/J mice. Control mice were inoculated intranasally with uninfected culture supernatant that had been either centrifuged on a renografin gradient or harvested at the sucrose interface. Mouse-to-mouse contamination was avoided by using individual, disposable equipment, including restrainers. Inoculated mice were subsequently bled every 2 or 3 weeks and sera were assayed for autoantibody development. Antiobiotic was delivered orally by lavage once per day as described.

### Example 3

### Characteristics of the Infectious Agent that Induces Anti-Centrosome Autoantibodies

To isolate the infectious agent responsible for centrosome autoantibody development, suspension homogenates were prepared from mouse spleens and were used to inoculate cultured mammalian cells. Plaques formed in cell monolayers inoculated with homogenates from mice with centrosome autoantibodies, but not from centrosome autoantibody-negative animals. For example, plaques formed by the infectious agent on monolayers of Vero cells. The infectious agent was plaque-purified and banded on renografin density gradients (1.19-1.20 g/cm3). Immunoblots showed strong autoreactivity develops in mice at two weeks to the centrosome proteins pericentrin and centriolin in mice (FIG. 11).

Plaques induced by the isolated infectious agent were indistinguishable from those observed with spleen homogenates and were atypical in many ways. Plaque formation was unusually prolonged (7-10 days post-infection compared to 1-4 days for most viruses) and was consistently observed in a wide range of host cells (fibroblasts, lymphocytes, and epithelial cells from human, monkey, and mouse). Infection of cultured cells occurred without significant cell tysis, except in agar plaque assays where cells lost could not be replaced by ingrowth of the surrounding monolayer. Several antibiotics prevented plaque formation (e.g., doxicyclin, ciprofloxacin, actinomycin D; FIG. 12) ruling out most viruses as infectious agents. The infectious agent appeared to be a membrane-bound particle as it was unable to induce plaque formation when treated with organic solvents or nonionic detergents.

Immunofluorescence of cells using antibodies generated against the infectious agent showed specific labeling of DNA-containing subcellular particles at the margins of plaques. Immunofluorescence detection of particles in cells at the margins of plaques was performed using antibodies raised against renografin fractions. At low magnification staining of material at margins of plaques defined by nuclei stained with DAPI was observed. Cells surrounding plaques did not stain. At higher magnification spherical particles ˜0.5 μm in diameter were observed. They co-localized with small DNA-containing particles labeled with DAPI next to the host cell nucleus. No staining was observed with preimmune serum or with mice that received culture supernatant from uninfected cells.

Plaque formation induced by the infectious agent in Vero cell monolayers is inhibited in the presence of the bacteriostatic antibiotics doxicyclin (Dox) and ciprofloxacin (Cip), and is similar to that seen in cultures that received no mycoplasma, or no mycoplasma plus antibiotics (FIG. 12). Assay sensitivity was 10 pfu/ml.

The banding pattern of the infectious agent from culture supernatants oil a renografin density gradient is shown in FIG. 13.

Electron microscopic examination of cells in plaques (FIG. 1A) and pellets of peak renografin gradient fractions (FIG. 1B) revealed morphologicaly homogeneous structures ˜0.45 mm in diameter that lacked cell walls and were closely apposed to the plasma membrane of host cells. Taken together, these features demonstrated that the agent was a mycoplasma.

### Example 4

### Identification of the Infectious Agent as Mycoplasma hyorhinis

Immunological and molecular biology methods were used to confirm the identity of the infectious agent as mycoplasma. Immunofluorescence microscopy using a monoclonal antibody specific for Mycoplasma hyorhinis variable lipoprotein (vip) 34 showed labeling of the margins of plaques containing the infectious agent. Nucleotide sequences obtained by random cloning of DNA isolated from renografin fractions containing the infectious agent exhibited a high degree of homology to Mycoplasma genes including the Mycoplasma hyorhinis-specific variable lipoprotein (vlp), 16S/23S rRNA intergenic spacer, pyruvate dehydrogenase, amino acid permease, and methylase (n=34 independent sequences); a low percentage of sequences (26%) showed no homology to mycoplasmas or any other NCBI database sequence. Additional mycoplasma sequences were obtained by PCR amplification of multiple mycoplasma genes. Using this approach sequences to 6 genes were obtained that were most homologous to mycoplasma, including the 16S rRNA gene, the putative transcription elongation factor (tuf), triosophosphate isomerase (tpi), a permease, a methylase and pyruvate dehydrogenase.

Because the procedure used to isolate the mycoplasma was not conventionally used for this purpose but rather for virus isolation, the microorganism was re-isolated using methods optimized for mycoplasmas. The microorganism was re-purified through three rounds of colony isolation on bacterial agar plates and growth in SP4 medium. The colony-purified material showed 100% homology to porcine Mycoplasma hyorhinis-specific vlp sequences by PCR analysis, thus confirming the identity of the original infectious agent as Mycoplasma hyorhinis. It was demonstrated that the purified mycoplasma was derived from mice and not from tissue culture cells, which can sometimes be contaminated with mycoplasmas. It was shown that cells used in plaque assays were mycoplasma-free as provided by the manufacturer (ATCC) and as demonstrated by PCR analysis using highly sensitive commercial primers (Stratagene) and others designed for purposes of this analysis. Moreover, it was demonstrated that mycoplasma sequences could be amplified from spleens and lungs of mice with spontaneous centrosome autoreactivity, but not from mice that tested negative for centrosome autoantibodies.

### Example 5

### Mycoplasma Induce Centrosome Autoantibodies in Naïve Mice

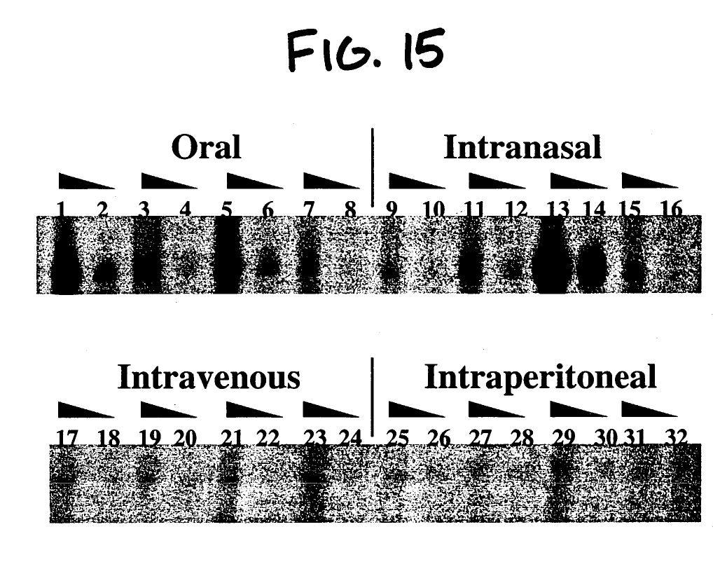

Next, it was tested whether introduction of the isolated mycoplasma fractions into naïve mice could induce centrosome autoantibody production. In an attempt to mimic the mode of infection in vivo, mycoplasma fractions were delivered into mice intranasally. It was found that high titer centrosome autoantibodies were produced in over half the mice (n>39, p=0.0016), while no mouse that received control inoculi tested positive (n>23, p=0.77) (FIG. 14).

Immunofluoresence was used to detect centrosome autoantibodies in the mice. Antibodies in sera from mice infected with Mycoplasma detected structures that costained with antibodies to the centrosome protein pericentrin. Mice that did not receive fractions containing mycoplasma did not stain centrosomes.

A more detailed analysis of centrosome autoreactivity in mice revealed that the mycoplasma-induced response was mainly of the IgG isotype. These isotypes were of the IgG1 and IgG2b subclasses, appeared as early as 2-3 weeks post-infection, and were well established by 6-8 weeks in all animals. The use of IgM-specific secondary antibodies revealed that IgM centrosome autoantibodies were present prior to IgGs, but did not increase in titer following mycoplasma infection. This demonstrated that high titer IgGs arose from centrosome-reactive IgMs by class switching and indicated that these IgMs were a part of the animal's natural antibody repertoire.

Mycoplasma-infected mice developed significantly higher titers of anti-centrosome IgG autoantibodies 21 weeks post-infection in contrast to uninfected mice (p=0.0016) or Mycoplasma-infected, autoantibody-negative mice (FIG. 14A). Anti-centrosome IgM autoantibody titers do not differ significantly in Mycoplasma-infected and uninfected mice 21 weeks post-infection (p=0.765, p>0.05) (FIG. 14B). Titers were determined by endpoint titration using the immunofluorescence assay and are expressed as the reciprocal of the serum dilution.

### Example 6

### Centrosome Autoantibody Development Requires an Established Mycoplasma Infection

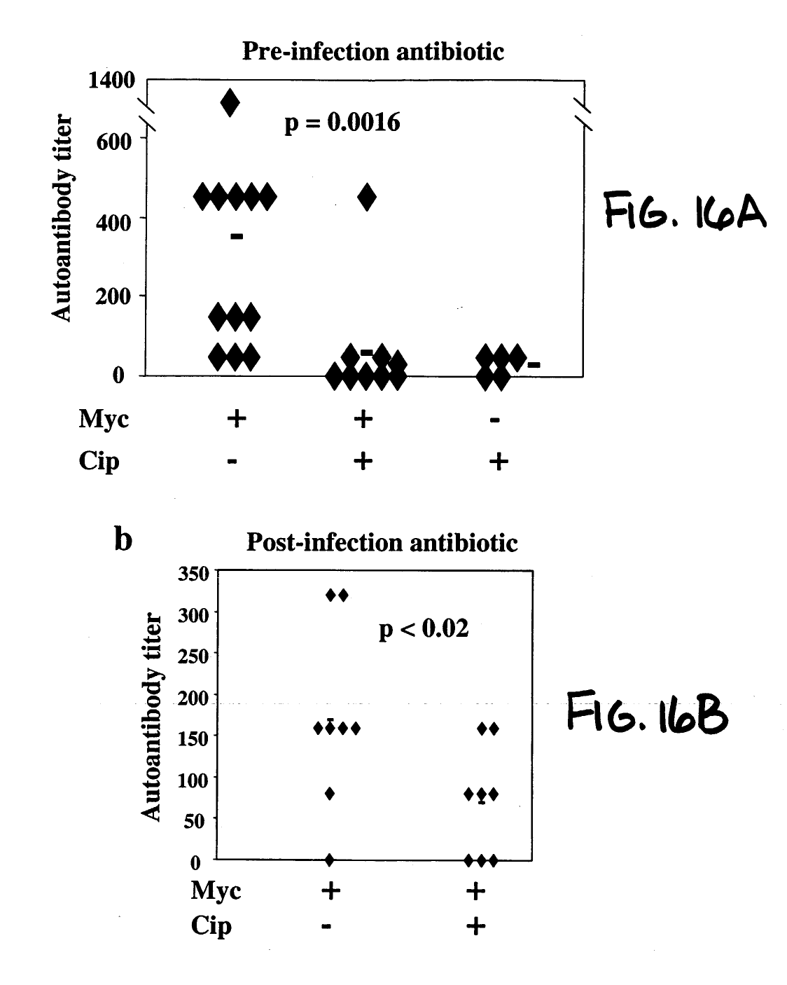

It was next addressed whether autoantibodies developed through passive exposure of the animal to mycoplasma antigens or if an established and persistent mycoplasma infection was required to induce and sustain autoreactivity. To test whether exposure to mycoplasma antigens was sufficient to generate autoantibodies, mice were inoculated with killed mycoplasma (FIG. 15). No autoantibody production was detected under these conditions. In addition, no autoantibody production was detected when live mycoplasma was introduced intraperitoneally or intravenously (FIG. 15). In contrast, the same dose of live mycoplasma introduced intranasally or orally induced strong autoreactivity in mice (FIG. 15). This demonstrated that the mode of delivery was important for autoantibody production and can be accomplished by infection of nasal epithelium, the most likely route utilized by this organism in vivo. Consistent with mycoplasma infection in this latter group of mice was the presence of anti-mycoplasma antibodies in their sera. By contrast, animals that did not receive mycoplasma tested negative for anti-mycoplasma antibodies. More significant was the observation that mice that received live mycoplasma by routes that were ineffective in generating autoantibodies (IV, 1P) or those that received mycoplasma intranasally but failed to develop autoantibodies did not develop mycoplasma antibodies. These results demonstrate that mycoplasma antibodies are induced only in animals that harbor mycoplasma and produce centrosome autoantibodies. This demonstrates a tight correlation between mycoplasma antibodies and centrosome autoantibodies and indicates that mycoplasma infection is a requirement for centrosome autoantibody production. Results with antibiotics further supported this observation.

### Example 7

### Antibiotics Prevent Autoantibody Production when Administered Before Infection and Reduce Production When Added after Infection

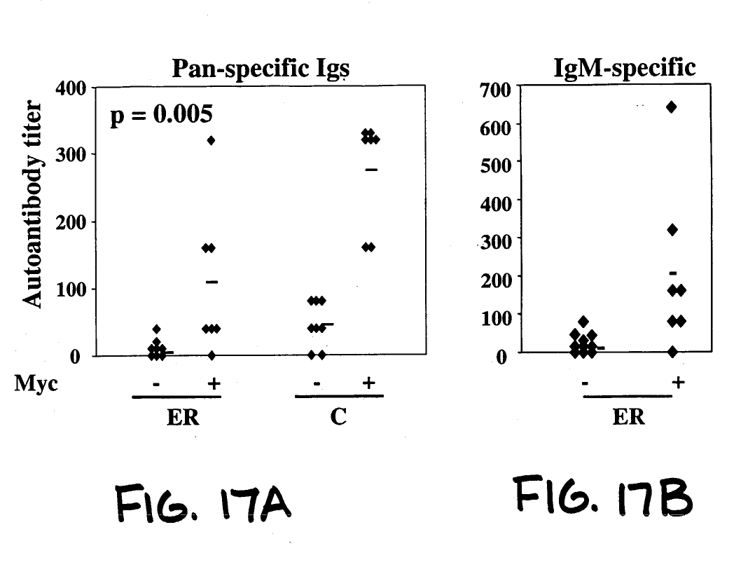

Testing was done to determine whether the pathological induction of autoantibodies by mycoplasma infection could be ameliorated by therapeutic intervention (FIG. 16). First, an attempt was made to prevent autoantibody production by pre-treating mice with the bacteriostatic agent ciprofloxacin prior to the addition of mycoplasma. It was found that autoantibody production was significantly inhibited in these mice, with autoantibody titers being indistinguishable from those in uninfected mice (FIG. 16A). This potent inhibition of autoantibody induction was observed at several times post-infection (3 and 6 weeks) and was significantly lower than that observed in mice that received no antibiotic (p 0.0013). Antibody induction was observed to be similar to that in uninfected mice (p=0.6).

Next, an attempt was made to reverse an established autoantibody response by administering antibiotics after mycoplasma infection. Infected mice were treated with ciprofloxacin for 3 weeks and examined for the presence centrosome autoantibodies. A three-week ciprofloxacin treatment of mice with high titer mycoplasma-induced centrosome autoantibodies significantly reduced autoantibody production in infected mice (p=0.026), while mice treated with solution lacking the antibiotic continued to exhibit strong centrosome autoantibody production (FIG. 16B). If antibiotic treatment was continued for longer periods of time (up to 6 weeks) a more significant reversal of autoantibody production was observed.

Anti-mycoplasma antibody titers declined in parallel with the reduction in autoantibody production. These results demonstrate that antibiotics prevent mycoplasma-induced autoantibodies and reverse previously-established autoantibody responses. These data also demonstrate that mycoplasma infection is required for establishing and sustaining autoantibody production in mammals, including mice and humans.

### Example 8

### The Centrosome-Specific Autoantibody Response Amplifies to Include Additional Intracellular Targets

In every animal that tested positive for autoantibodies, exclusive autoreactivity to centrosomes was observed, and never to other structures or organelles. In most cases, multiple components of a single organelle (the centrosome) were targeted in this initial response (pericentrin, centriolin). These observations demonstrated that centrosomes or centrosome antigens or both initiated the autoantibody response in mycoplasma-infected mice.

Next, it was addressed whether the initial centrosome autoantibody response could subsequently spread to include reactivity to other intracellular organelles and structures. This phenomenon, known as antigenic spreading, has been implicated in the generation of complex multi-target autoantibody responses in autoimmune diseases (Craft and Fatenejad, Arthritis Rheum. 40:1374-1382, 1997). Mice were examined at later times after mycoplasma infection to determine whether additional autoantibodies were generated. Beginning at approximately 12 weeks post-infection, autoantibodies to another intracellular structure observed in infected mice were detected. Immunofluorescence co-localization of mycoplasma-induced autoantibodies with antibodies to calnexin and binding protein of the endoplasmic reticulum (Bip) demonstrated that this structure was the endoplasmic reticulum (ER). Autoantibodies to the ER were exclusively of the IgM isotype. They did not class switch to IgG like the centrosome autoantibodies and they were lower titer than anti-centrosome autoantibodies. In some animals, autoantibodies to cytoskeletal elements (intermediate filaments) also appeared 12 weeks after infection.

These results demonstrate that mice elicit complex autoantibody responses following mycoplasma infection that appear to be initiated by centrosomes. Multiple components of centrosomes are targeted (pericentrin, centriolin) prior to involvement of other cellular organelles/structures. This demonstrates that complex multi-molecular autoreactive responses can be generated that are specific for a single organelle but ultimately spread to other cellular structures. This observation the occurrence of antigenic spreading where autoreactivity spreads between components of the same structure prior to involving other structures.

### Example 9

### Centrosome Autoantibody Response Amplifies to Involve Endoplasmic Reticulum-Associated Autoantigens

Several weeks after the appearance of centrosome autoreactivity in Mycoplasma-infected mice, autoantibodies to a second cytoplasmic structure were detected by immunofluorescence. The autoantibodies co-localized with the endoplasmic reticulum protein calnexin. Titers of the ER-associated autoantibodies in Mycoplasma-infected mice were significantly higher than in uninfected mice (p=0.005) (FIG. 17A). Data were obtained using an antibody that recognizes all immunoglobulin isotypes and both heavy and light chains. Results were representative of 3 independent experiments. Unlike centrosome autoantibodies, those to the ER-associated autoantigen are of IgM: isotype, and have significantly higher titers than uninfected mice (FIG. 17B).

### Example 10

### Assay to Determine Substances Useful in Treating Mycoplasma hyorhinis Infection

Using a murine model of Mycoplasma hyorhinis infection, mice are treated with the therapeutic agents of the invention (e.g., antibiotics, antibodies, vaccines, antisense nucleic acids, ribozymes, etc.). Then, appropriate assays such as immunofluorescence, RIA, ELISA, Western blot, plaque formation, mouse re-infection, gradient isolation, PCR, or Southern blots for DNA are employed to determine which substances are useful in treating Mycoplasma hyorhinis infection. This process can be carried once, or over multiple rounds, to improve successively the efficacy of the substances for treating M. hyorhinis infection.

### Example 10

### Treatment of Scleroderma with Antibiotics

Clindamycin is administered to a patient presenting scleroderma caused by an infection by Mycoplasma hyorhinis. On the first day of treatment, 300 mg of clindamycin is administered to the patient intravenously in 250 cc 0.9 saline drip that delivers the full dosage over a period of 60 minutes. The same treatment is administered on the second day. On the third day, the dosage of clindamycin is doubled to 600 mg delivered by the same method as on days 1 and 2. This is repeated on day 4. On days 5, 6, and 7, the dosage is again raised, this time to 700 mg, and delivered in the same 60 minute 250 cc 0.9 saline drip. Administration of clindamycin is discontinued after day 7.

### Example 11

### Production of a Mouse Model of Scleroderma

The invention encompasses the production of mammalian models (e.g., a mouse, rat, pig, goat, sheep, cow etc.) of scleroderma and other autoimmune diseases.

To produce a mouse model of scleroderma, a mouse is infected with Mycoplasma hyorhinis designated ATCC #PTA-3681 via intranasal delivery. The detection methods of the invention are used to detect M. hyorhinis designated ATCC #PTA-3681 infection in the mouse to whom the mycoplasma was delivered. Once such infection is detected, the model mouse can be used for a variety of purposes related to the invention, Such as assays to determine substances useful in treating or preventing mycoplasma infection.

### Deposit Made Under the Budapest Treaty

Applicants have deposited the subject cultures with the American Type Culture Collection (ATCC), 10801 University Blvd., Manassas, Va. 20110-2209, U.S.A. The deposit was received by the ATCC on Aug. 30, 2001. The viability of the culture was tested and confirmed on Oct. 16, 2001. The subject cultures deposited with the ATCC are taken from the same deposit maintained by Steven Doxsey at the University of Massachusetts since prior to the filing date of this application. The ATCC deposit number is PTA-3681.

The subject cultures have been deposited under conditions that assure that access to the cultures will be available during the pendency of the patent application disclosing them to one determined by the Commissioner of Patents and Trademarks to be entitled thereto under 37 C.F.R. 1.14 and 35 U.S.C. 122. The deposits are available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of the deposits, and in any case, for a period of at least 30 (thirty) years after the date of deposit or for the enforceable life of any patent which may issue disclosing the cultures plus five years after the last request for a sample from the deposit. The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid sequence comprising any one of SEQ ID NOS:1-22.

2. An isolated polypeptide encoded by a nucleic acid sequence comprising any one of SEQ ID NOS:1-22.

3. An isolated Mycoplasma hyorhinis designated ATCC #PTA-3681.

4. An isolated tissue culture comprising the isolated Mycoplasma hyorhinis of claim 3.

5. A mammalian model of autoimmune disease, wherein the mammal is infected with a sufficient amount of isolated Mycoplasma hyorhinis to produce symptoms of autoimmune disease.

6. The model of claim 5, wherein the autoimmune disease is systemic sclerosis.

7. The model of claim 5, wherein the Mycoplasma hyorhinis is designated ATCC #PTA-3681.

8. The model of claim 5, wherein the mammal is a mouse or a pig.

9. A method of producing a mammalian model of systemic sclerosis, the method comprising exposing a mammal to an amount of Mycoplasma hyorhinis sufficient to induce infection, wherein the infection induces symptoms of systemic sclerosis.

10. The method of claim 9, wherein the Mycoplasma hyorhinis is designated ATCC #PTA-3681.

11. The method of claim 9, wherein the mammal is a mouse or a pig.

12. A method of treating an animal having symptoms of systemic sclerosis, the method comprising:
(a) obtaining a sample from the animal;
(b) testing the sample for the presence of Mycoplasma hyorhinis; and, if the sample contains Mycoplasma hyorhinis
(c) administering to the animal an amount of a composition effective to treat the Mycoplasma hyorhinis within the animal, thereby reducing the severity of the systemic sclerosis.

13. The method of claim 12, wherein the Mycoplasma hyorhinis is designated ATCC #PTA-3681.

14. The method of claim 12, wherein the composition comprises an antibody that specifically binds to Mycoplasma hyorhinis.

15. The method of claim 12, wherein the composition comprises an aptamer that specifically binds to Mycoplasma hyorhinis.

16. The method of claim 12, wherein the composition comprises an antisense nucleic acid that specifically hybridizes to an mRNA complementary to any one of SEQ ID NOs:1-22.

17. The method of claim 12, wherein the composition comprises a ribozyme that specifically cleaves an mRNA transcript of any one of SEQ ID NOs:1-22.

18. The method of claim 12, wherein the composition comprises an siRNAi or dsRNA.

19. The method of claim 12, wherein the composition comprises an antibiotic.

20. The method of claim 19, wherein the antibiotic is chloramphenicol, tetracycline, ciprofloxacin, or doxicycline.

21. The method of claim 12, wherein the composition comprises a vaccine.

22. The method of claim 21, wherein the vaccine is a DNA vaccine.

23. The method of claim 12, wherein the animal is a mammal.

24. The method of claim 23, wherein the mammal is a mouse, rat, dog, cat, pig, sheep, goat, cow, horse, or human.

25. The method of claim 12, wherein the animal is a bird.

26. A method for treating systemic sclerosis in a subject, the method comprising:
(a) providing an organism, tissue, or cells infected with Mycoplasma hyorhinis; and
(b) administering to the subject or the subject's tissue an amount of a composition effective to inhibit, reduce, or eliminate the Mycoplasma hyorhinis within the animal, thereby reducing the severity of the systemic sclerosis.

27. The method of claim 26, wherein the Mycoplasma hyorhinis is designated ATCC #PTA-3681.

28. A method of detecting Mycoplasma hyorhinis in a sample, the method comprising:
(a) obtaining a sample; and
(b) determining whether any nucleic acid sequence in the sample is identical to a nucleotide sequence of SEQ ID NOs:1-22, wherein sequence identity indicates the presence of Mycoplasma hyorhinis in the sample.

29. The method of claim 28, wherein the Mycoplasma hyorhinis is designated ATCC #PTA-3681.

30. A method of detecting Mycoplasma hyorhinis in a sample, the method comprising:
(a) obtaining a sample from an organism; and
(b) detecting in the sample the presence of antibodies that specifically bind to Mycoplasma hyorhinis, wherein the presence of the antibodies indicates the presence of Mycoplasma hyorhinis in the sample.

31. The method of claim 30, wherein the Mycoplasma hyorhinis is designated ATCC #PTA-3681.

32. An isolated antibody that specifically binds to Mycoplasma hyorhinis designated ATCC #PTA-3681.

33. A method for detecting in a biological sample an antibody that specifically binds to all antigenic site of a Mycoplasma hyorhinis, the method comprising:
(a) contacting the sample with an antigen isolated from a Mycoplasma hyorhinis; and
(b) measuring the formation of an antigen-antibody complex, wherein the presence of a complex indicates the presence of the antibody.

34. The method of claim 33, wherein the Mycoplasma hyorhinis is designated ATCC #PTA-3681.

35. The method of claim 33, wherein the antigen is isolated from the Mycoplasma hyorhinis by sonication.

36. The method of claim 33, wherein the antigen is isolated from the Mycoplasma hyorhinis by detergent extraction.

37. A method for diagnosing systemic sclerosis in a subject, the method comprising:
(a) obtaining a biological sample from the subject;
(b) contacting the sample with antigen of Mycoplasma hyorhinis; and
(c) measuring the formation of antigen-antibody complex, wherein the presence of a complex indicates that the subject has systemic sclerosis.

38. The method of claim 37, wherein the Mycoplasma hyorhinis is designated ATCC #PTA-3681.

39. The method of claim 37, wherein the sample is skin tissue.

40. The method of claim 39, wherein the skin tissue is lesioned.

41. A composition comprising purified antigen from Mycoplasma hyorhinis designated ATCC #PTA-3681.

42. A method of generating antibodies specific for Mycoplasma hyorhinis designated ATCC #PTA-3681, the method comprising:
(a) providing antigen from Mycoplasma hyorhinis designated ATCC #PTA-368 1; and
(b) introducing into an animal a composition comprising antigen from Mycoplasma hyorhinis designated ATCC #PTA-3681, wherein antibodies specific for Mycoplasma hyorhinis designated ATCC #PTA-3681 are generated.
